SEQUENCE LISTING (41 sequences)

SEQ ID NO: 1 - RNA, 7 nt, Homo sapiens
aauguuu

SEQ ID NO: 2 - RNA, 22 nt, Homo sapiens
caauguuuccacagugcaucac

SEQ ID NO: 3 - RNA, 21 nt, Homo sapiens
aauguuuccacagugcaucac

SEQ ID NO: 4 - RNA, 18 nt, Homo sapiens
aauguuuccacagugcau

SEQ ID NO: 5 - RNA, 15 nt, Homo sapiens
aauguuuccacagug

SEQ ID NO: 6 - RNA, 12 nt, Homo sapiens
aauguuuccaca

SEQ ID NO: 7 - RNA, 22 nt, Homo sapiens
caauguuuccacagugcaucac

SEQ ID NO: 8 - RNA, 19 nt, Homo sapiens
caauguuuccacagugcau

SEQ ID NO: 9 - RNA, 16 nt, Homo sapiens
caauguuuccacagug

SEQ ID NO: 10 - RNA, 13 nt, Homo sapiens
caauguuuccaca

SEQ ID NO: 11 - RNA, 8 nt, Artificial Sequence
  A synthetic sequence
nauguuun

SEQ ID NO: 12 - RNA, 9 nt, Artificial Sequence
  A synthetic sequence
nnauguuun

SEQ ID NO: 13 - RNA, 9 nt, Artificial Sequence
  A synthetic sequence
nanuguuun

SEQ ID NO: 14 - RNA, 9 nt, Artificial Sequence
  A synthetic sequence
naanguuun

SEQ ID NO: 15 - RNA, 9 nt, Artificial Sequence
  A synthetic sequence
naaunuuun

SEQ ID NO: 16 - RNA, 9 nt, Artificial Sequence
  A synthetic sequence
naaugnuun

SEQ ID NO: 17 - RNA, 9 nt, Artificial Sequence
  A synthetic sequence
naaugunun

SEQ ID NO: 18 - RNA, 9 nt, Artificial Sequence
  A synthetic sequence
naauguunn

SEQ ID NO: 19 - DNA, 70 bp, Homo sapiens
ctgtggtgcattgtagttgcattgcatgttctggtggtacccatgcaatgtttccacagtgcatcacaga

SEQ ID NO: 20 - RNA, 23 nt, Homo sapiens
ccucccucacuguggggcauuuc

SEQ ID NO: 21 - RNA, 23 nt, Artificial Sequence
  A synthetic sequence
ccucccucacugugggcgauuuc

SEQ ID NO: 22 - RNA, 22 nt, Homo sapiens
caauguuuccacagugcaucac

SEQ ID NO: 23 - RNA, 23 nt, Homo sapiens
gccccagggucuggaaugcaaaa

SEQ ID NO: 24 - RNA, 23 nt, Artificial Sequence
  A synthetic sequence
gccccagggucuggaaaccaaaa

SEQ ID NO: 25 - RNA, 21 nt, Homo sapiens
gugcauuguaguugcauugca

SEQ ID NO: 26 - RNA, 26 nt, Homo sapiens
agaugaccuuaucgggugcaauacua

SEQ ID NO: 27 - RNA, 26 nt, Mus musculus
agcugaccucaucgggugcaauacua

SEQ ID NO: 28 - RNA, 26 nt, Homo sapiens
uuccacucccacuugggcauuuugga

SEQ ID NO: 29 - RNA, 26 nt, Mus musculus
uuucacccccacuugggcauuuugga

SEQ ID NO: 30 - RNA, 22 nt, Homo sapiens
caauguuuccacagugcaucac

SEQ ID NO: 31 - RNA, 22 nt, Mus musculus
caauguuuccacagugcaucac

SEQ ID NO: 32 - RNA, 21 nt, Oryctolagus cuniculus
caauguuuccacagugcaucc

SEQ ID NO: 33 - RNA, 20 nt, Ophiophagus hannah
caauguuucugcagugcagu

SEQ ID NO: 34 - RNA, 23 nt, Cavia porcellus
caauucguuuccacagugcauca

SEQ ID NO: 35 - RNA, 19 nt, Capra hircus
caauguuuccacagugcaa

SEQ ID NO: 36 - RNA, 21 nt, Dasypus novemcinctus
caauguguccacagugcaucc

SEQ ID NO: 37 - RNA, 21 nt, Oreochromis niloticus
caaugugucugcagugcagua

SEQ ID NO: 38 - RNA, 21 nt, Salmo salar Linnaeus
caaugugucugcagugcagua

SEQ ID NO: 39 - RNA, 21 nt, Xenopus laevis
caaugugccugcagugcaaca

SEQ ID NO: 40 - RNA, 21 nt, Pteropus alecto
caguguuuccacagugcauca

SEQ ID NO: 41 - RNA, 20 nt, Ornithorhynchus anatinus
caaugccccugcagugcaau

(19) United States
(12) Patent Application Publication
(10) Pub. No.: US 2024/0368606 A1
(43) Pub. Date: Nov. 7, 2024

(54) USE OF microRNA MIMICS TO INHIBIT OR TREAT LIVER DISEASE

(71) Applicant: CORNELL UNIVERSITY, Ithaca, NY (US)

(72) Inventors: Seyed Hani Najafi Shoushtari, New York, NY (US); Vimal Ramachandran, Doha (IN)

(21) Appl. No.: 18/562,233

(22) PCT Filed: May 18, 2022
(86) PCT No.: PCT/US2022/029884

§ 371 Date: Nov. 17, 2023

Related U.S. Application Data
(60) Provisional application No. 63/189,807, filed on May 18, 2021.

(51) Int. Cl. C12N 15/113; A61P 1/16

(52) U.S. Cl. CPC C12N 15/1137; A61P 1/16; C12Y 304/21061; C12N 2310/141; C12N 2310/315; C12N 2310/321; C12N 2310/322; C12N 2310/3231

(57) ABSTRACT

Described herein are compositions and methods for treatment of liver conditions and diseases. The compositions and methods include use of microRNAs, including miR-33a-3p, that can reduce the incidence and progression of chronic or non-chronic liver conditions and liver diseases, such as hepatic steatosis, non-alcoholic fatty liver disease (NAFLD), nonalcoholic steatohepatitis (NASH), and the like.

### CROSS-REFERENCE TO RELATED APPLICATION

This application claims the priority of U.S. provisional application Ser. No. 63/189,807, filed May 18, 2021, the disclosure of which is incorporated herein by reference in its entirety.

### INCORPORATION BY REFERENCE OF SEQUENCE LISTING PROVIDED AS A TEXT FILE

A Sequence Listing is provided herewith as a text file, “2238988.txt,” created on May 16, 2022, and having a size of 8,192 bytes. The contents of the text file are incorporated by reference herein in their entirety.

### BACKGROUND

Sterol regulatory element-binding protein-2 (SREBP-2) directed transcription of low-density lipoprotein (LDL) receptor is involved in the removal of atherogenic LDL from circulation and the maintenance of cholesterol homeostasis (Hua et al., 1993; Horton et al., 1998; Horton et al., 2003). At the post-translational level, however, LDLR-mediated cholesterol uptake is limited by SREBP-2- and liver X receptor (LXR)-induced counter-mechanisms involving activation of proprotein convertase subtilisin/kexin type 9 protease (PCSK9) and the E3 ubiquitin ligase IDOL-promoted degradation of LDLR (Horton et al., 2003; Abifadel et al., 2003). However, the coordinated cellular mechanisms that restrict or prevent LDLR from being degraded upon transcription remain obscure.

In metazoans, cholesterol levels are tightly regulated by opposing but complementary regulatory circuits. SREBP-2 and the nuclear liver X receptor alpha (LXRα) transcriptionally control genes that integrate cholesterol biosynthesis, uptake, and efflux for homeostasis (Madison, 2016; Wang & Tontonoz, 2018). While SREBP-2 preferentially directs cellular events towards higher cholesterol content, LXRα mainly activates genes in the cholesterol transport pathway, conversion into bile acids, secondary to suppression of sterol synthesis. In response to hepatic cellular cholesterol demand, SREBP-induced expression of low-density lipoprotein (LDL) receptor (LDLR) forms a critical step in boosting intracellular cholesterol levels and clearance of pro-atherogenic LDL particles by LDLR-mediated endocytosis (Goldstein & Brown, 2009). Excessive cholesterol levels, on the other hand, are mainly remedied by LXRα activity among other genes through ATP-binding cassette A1 (ABCA1)-mediated cholesterol efflux for transport to the periphery (Phillips, 2018; Navab et al., 2011). Thereby, coordinated counteracting mechanisms must exist to control sterol-sensitive LDLR and ABCA1 expression levels in states of SREBP-2 and LXR activation, beyond regulation by sterol content. For instance, microRNAs located within SREBP introns, miR-33a-5p in SREBP2 and miR-33b-5p in SREBP1c, antagonize the LXR pathway in support of the SREBP function by direct inhibition of ABCA1, a canonical LXR target gene, at post-transcriptional level (Najafi-Shoushtari et al., 2010; Marquart et al., 2010).

Similarly, the SREBP-dependent transcription of proprotein convertase subtilisin/kexin type-9 (PCSK9) and the LXR-induced expression of E3-ubiquitin ligase IDOL, are part of two major regulatory branches that limit LDLR expression at post-translational level through lysosomal degradation (Page & Watts, 2018; Zhang et al., 2012). However, how LDLR expression is maintained and prevented from degradation upon SREBP-mediated transcription remains highly elusive.

### SUMMARY

As disclosed herein, the effect of miR-33a on LDL-uptake was assessed. Herein it is shown that miR-33a extends the regulatory arm of the SREBP-2 pathway and acts to control cholesterol efflux, as well as promoting cholesterol uptake by direct inhibition of LDLR-degrading PCSK9 and IDOL proteins. Also shown is that both strands of microRNA 33a (miR-33a) duplex, encoded within SREBP-2, cooperatively act to promote LDLR expression through direct targeting of PCSK9 and IDOL. In humans and mice, antisense-mediated silencing of miR-33a-3p/5p led to a concomitant decrease in LDLR protein levels and restrained LDL-cholesterol uptake without a change in LDLR mRNA. Conversely, miR-33a-3p/5p expression under sterol-deprivation and LXR-induced conditions elevated LDLR expression dependent of PCSK9 and IDOL. Although miR-33a-5p was identified as a major direct inhibitor of ATP-binding cassette A1 (ABCA1) (Zelcer et al., 2009), increased expression of miR-33a-3p was found in hepatocytes and macrophages to strand specifically elevate the expression of ABCA1 and increased cholesterol efflux. Liver-targeted delivery of miR-33a-3p mimics into mouse models of diet-induced obesity resulted in reduced hepatic and circulating PCSK9 levels, significantly lowered LDL, and ameliorated hepatic steatosis secondary to increased VLDL secretion and genes involved in fatty acid oxidation. These findings reveal a compensatory control mechanism for PCSK9 and IDOL expression and extend miR-33a complementary function in mutually exclusive regulation of LDLR and ABCA1 by SREBP-2 and LXR. miR-33a-3p mimics represent alternative therapeutic inhibitors of PCSK9 and LDL-cholesterol with pleiotropic effects on reducing hypercholesterolemia and steatohepatitis.

In one embodiment, the disclosure provides a method to prevent, inhibit or treat liver disease in a mammal, comprising: administering to a mammal in need thereof an effective amount of a composition comprising a nucleic acid sequence comprising a seed region of miRNA-33a-3p, e.g., useful as a guide. In one embodiment, the mammal is a human. In one embodiment, the disease is steatosis, non-alcoholic fatty liver disease (NAFLD), or nonalcoholic steatohepatitis (NASH). In one embodiment, the mammal has alcohol fatty liver disease or chronic liver disease. In one embodiment, the composition comprises liposomes. In one embodiment, the liposomes comprise or more of DC-cholesterol, 1,2-dioleoyl-sn-glycero-3-phosphoethanolamine (DOPE), an ionizable cationic lipid, e.g., 2,2-dilin-oleyl-4-(2-dimethylaminoethyl)-[1,3]-dioxolane) or 1,2-dilinoleyloxy-N,N-dimethyl-3-aminopropane, or a lipidoid (which contains tertiary amines). In one embodiment, the composition comprises nanoparticles, e.g., formed of lipids and/or non-lipid biocompatible materials. In one embodiment, the composition, liposomes and/or nanoparticles, is targeted to the liver, e.g., comprises collagen type VI receptor, mannose-6-phosphate, galactose or asialoglycoprotein. In one embodiment, the composition is systemically administered. In one embodiment, the composition is orally administered. In one embodiment, the composition is injected. In one embodiment, the amount reduces total cholesterol levels in blood of the mammal. In one embodiment, the amount reduces LDL levels in the mammal. In one embodiment, the amount alters triglycerides e.g., VLDL-associated triglycerides, levels in the mammal. In one embodiment, the seed region comprises 5′AAUGUUU3′ or 5′AATGTTT3′. In one embodiment, the nucleic acid sequence is less than 100, 50, 30, or 25 bases in length. In one embodiment, the nucleic acid sequence is greater than 10 bases in length. In one embodiment, the composition comprises single stranded RNA comprising the seed region. In one embodiment, the composition comprises RNA comprising a hairpin-loop structure. In one embodiment, the composition comprises double stranded nucleic acid comprising the seed region. In one embodiment, the RNA or one strand of the double stranded nucleic acid comprises an antisense sequence of miRNA-33a-3p, e.g., a passenger strand. In one embodiment, the passenger strand includes modified nucleotides that enhance degradation of the single stranded passenger strand. In one embodiment, the miRNA includes modified nucleotides that inhibit degradation, e.g., modifications in the base and/or sugar moiety. In one embodiment, the RNA or the one strand is less than 100, 70, 50 or 25 bases in length. In one embodiment, the RNA or the one strand is greater than 10 bases in length. In one embodiment, the RNA or the one strand comprises non-native nucleotides, e.g., a modified nucleobase, modified phosphate group or a modified sugar. In one embodiment, the amount of the nucleic acid sequence is about 0.01 mg/kg to about 100 mg/kg. In one embodiment, the amount of the nucleic acid sequence is about 0.05 mg/kg to about 10 mg/kg, e.g., about 0.5 mg/kg to 5 mg/kg such as about 1 to 2 mg/kg. In one embodiment, the amount of the nucleic acid sequence is about 10 mg/kg to about 75 mg/kg, e.g., about 25 mg/kg to about 50 mg/kg.

Further provided is a liver targeted, lipid composition comprising a nucleic acid sequence comprising a seed region of miRNA-33a-3p. In one embodiment, the composition comprises nanoparticles. In one embodiment, the composition comprises complexes comprising one or more distinct lipids including a cationic lipid and a nucleic acid sequence comprising a seed region of miRNA-33a-3p. In one embodiment, at least one of the lipids comprises a liver targeting molecule, e.g., Gal-NAc, such as a liver targeted molecule conjugated to the lipid.

Also provided is a liver targeted molecule comprising a nucleic acid sequence comprising a seed region of miRNA-33a-3p, e.g., Gal-NAc conjugated to a nucleic acid sequence comprising a seed region of miRNA-33a-3p.

### BRIEF DESCRIPTION OF FIGURES

FIG. 1A-1J illustrate that miR-33a promotes Low Density Lipoprotein Receptor (LDLR) expression and Low Density Lipoprotein (LDL) uptake. FIG. 1A is a schematic diagram illustrating that the chromosomal location of miR-33a is within an intron of the SREBP-2 gene. The sequence shown for miR33a is CTGTGGTGCATTGTAGTTGCATTGCATGTTCTGGTGGT ACCCATGCAATG TTTCCACAGTGCATCACAGA (SEQ ID NO:19). As shown, miR-33a is transcribed with SREBP-2. FIG. 1B-1E illustrate expression of miR-33a and SREBP2/LXR targets under different media conditions in HepG2 cells. FIG. 1B graphically illustrates miR-33a-3p and miR-33a-5p expression relative to miR-423-3p as determined by quantitative RT-PCR in HepG2 cells subjected to FBS (normal), low sterol (LPDS+simvastatin+sodium mevalonate), or FBS+GW3965 (LXR-activating) media conditions. FIG. 1C graphically illustrates ABCA1 (ATP-binding cassette A1) and IDOL (“Inducible Degrader of the LDL receptor”) gene expression relative to HMBS as determined by quantitative RT-PCR in HepG2 cells subjected to the same media conditions described for FIG. 1B. FIG. 1D graphically illustrates SREBP2, LDLR, PCSK9, and HMGCR gene expression relative to HMBS as determined by quantitative RT-PCR in HepG2 cells subjected to the same media conditions described for FIG. 1B. FIG. 1E shows a western blot, illustrating protein expression of ABAC1, LDLR, and PCSK9 proteins in HepG2 cells subjected to the same media conditions described for FIG. 1B. FIG. 1F is a schematic diagram illustrating the effects of SREBP-2 as well as the hypothesized effects of miR-33a-3p and miR-33a-5p on LDLR expression. FIG. 1G graphically illustrates that miR-33a-3p and miR-33a-5p have little or no effects on LDLR mRNA expression. The plot reflects densitometric analysis of LDLR band from (n=3) independent experiments. PC refers to a precursor control (inactive). FIG. 1H illustrates that antisense-mediated inhibition of miR-33a-3p and miR-33a-5p reduces LDLR protein expression levels, while miR-33a-3p and miR-33a-5p over-expression elevates LDLR protein expression levels in HepG2 cells and human primary hepatocytes. AC refers to an antisense control (inactive). All blots are representative of at least 3 independent experiments. FIG. 1I shows immunofluorescence images of HepG2 cells showing Dil-LDL (red in the original), LDLR (green in the original) and DAPI (blue in the original) treated with Precursor control (PC, inactive), miR-33a-3p, or miR-33a-5p for 48 hours. The graph to the right illustrates mean LDLR fluorescence intensity for PC, miR-33a-3p and miR-33a-5p treated cells. FIG. 1J graphically illustrates Dil-LDL uptake in HepG2 cells in media having lipid-depleted serum treated with miR-33a-3p and miR-33a-5p antisense (left plot) or Dil-LDL uptake in HepG2 cells in media having lipid-depleted serum treated with miR-33a-3p or miR-33a-5p (right plot). *P<0.05, **P<0.01. Error bars represent in this and subsequent figures the mean SD.

FIG. 2A-2J illustrate that miR-33a-5p and miR-33a-3p directly inhibit PCSK9 expression and function. FIG. 2A is a schematic diagram illustrating post-transcriptional and transcription effects on PCSK9 and LDLR expression. FIG. 2B shows western blots illustrating mature PCSK9 protein expression in the liver cell line HepG2 and in human primary hepatocytes under lipid depleted conditions after miRNA inhibition (Anti-33a) or miRNA overexpression (miR-33a). FIG. 2C graphically illustrates PCSK9 mRNA expression in HepG2 cells under the same conditions described in FIG. 2B. Actin was used as a loading control. FIG. 2D shows western blots illustrating that antisense-antagonism and overexpression of miR-33a-3p and miR-33a-5p respectively increases and decreases PCSK9 secretion into the media. FIG. 2E shows PCSK9 3′-UTR and PCSK9 coding sequences depicting non-canonical miR-33a-3p and miR-33a-5p binding sites. The miR-33a-3p and miR-33a-5p binding sites in the PCSK9 sequences were altered by site-directed mutagenesis to generate the mutant PCSK9 sequences that are also shown. The miR-33a seed regions are highlighted in bold black letters. The wild type human PCSK9 3′-UTR sequence shown is CCUCCCUCACUGUGGGGCAUUUC (SEQ ID NO:20), and the mutant human PCSK9 3′-UTR sequence CCUCCCUCACUGUGGGCGAUUUC (SEQ ID NO: 21) is also shown, with the mutations highlighted. The alignment of the PCSK9 3′-UTR is also shown with the human miR-33a-3p, having the 5′ to 3′ sequence: CAAUGUUUCCACAGUGCAUCAC (SEQ ID NO:22) with the seed region highlighted. The SEQ ID NO:22 has the sequence of the active strand of the miR-33a-3p. The lower sequence alignments illustrate a wild type human PCSK9 partial coding region sequence, GCCCCAGGGUCUGGAAUGCAAAA (SEQ ID NO:23), and a mutant human PCSK9 partial coding region sequence, GCCCCAGGGUCUGGAAACCAAAA (SEQ ID NO:24). The lower alignment also shows the alignment of partial human PCSK9 coding regions with miR-33a-5p, having 5′ to 3′ sequence GUGCAUUGUAGUUGCAUUGCA (SEQ ID NO:25 for miR-33a-5p) with the seed region highlighted. FIG. 2F graphically illustrates luciferase activity when the wild type and mutant PCSK9 3′-UTR constructs shown in FIG. 2E are expressed at the same time as miR-33a-3p. Luciferase activity was quantified from HEK293T cells transfected with a luciferase reporter containing wild-type or mutated miR-33a-3p binding site of human PCSK9 3′-UTR, co-transfected with the indicated miRNA precursors. As illustrated, miR-33a-3p reduces luciferase activity when the wild type PCSK9 3′-UTR is linked to luciferase, but not when the mutant PCSK9 3′-UTR is linked to luciferase. FIG. 2G shows western blots illustrating flag-tagged human PCSK9 protein expression from the wild type and mutant PCSK9 constructs, having the wild type and mutant miR-33a-5p binding sites shown in FIG. 2E. PCSK9 constructs were co-transfected with precursor miR-control (PC) or miR-33a-5p. As illustrated, miR-33a-5p reduced expression of flag-tagged human PCSK9 protein with the wild-type (WT) protein coding region but not with the mutant flag-tagged human PCSK9 protein coding region. PCSK9 constructs were co-transfected with precursor miR-control (PC) or miR-33a-5p. FIG. 2H shows western blots illustrating flag-tagged mouse PCSK9 protein expression from the wild type and mutant mouse PCSK9 constructs, having the wild type and mutant miR-33a-3p or miR-33a-5p binding sites. The wild type and mutant PCSK9 constructs were co-transfected with precursor miR-control (PC), miR-33a-3p or miR-33a-5p. As illustrated, miR-33a-3p and miR-33a-5p reduce expression of flag-tagged mouse PCSK9 protein with wild-type (WT) but not in the mutant PCSK9 protein coding regions. Precursor miR-control (PC, inactive). FIG. 2I shows a western blot illustrating mouse LDLR and mouse PCSK9 protein expression in the presence of antisense PCSK9 as well as antisense miR-33a-3p or antisense miR-33a-5p when cholesterol is not present. Little or no PCSK9 protein is detectable. LDLR expression is rescued by siRNA-mediated repression of PCSK9 and antisense inhibition of miR-33a. FIG. 2J illustrates expression of flag-tagged mouse PCSK9 protein from transfected plasmid constructs harboring wild-type (WT) or mutated (Mutant) miR-33a-5p or miR-33a-3p binding sites within mouse PCSK9 CDS region, co-transfected with miR-33a-3p, miR-33a-5p, a scrambled antisense miR-control (AC, inactive).

FIG. 3A-3J illustrate that miR-33a-5p and miR-33a-3p directly inhibit IDOL expression. FIG. 3A is a schematic illustrating interactions between LXR-agonists, IDOL, miR-33a and LDLR. FIG. 3B graphically illustrates relative mRNA expression of ABCA1 and IDOL in HepG2 cells maintained in LPDS (Lipoprotein Deficient Serum) media, FBS (normal conditions) media, and media with an LXR-agonist (GW3965, GW) FIG. 3C graphically illustrates that both miR-33a strands repress IDOL mRNA expression in HepG2 cells under various media conditions involving LPDS (Lipoprotein Deficient Serum), FBS (normal conditions) media, and media with an LXR-agonist (GW3965). FIG. 3D graphically illustrates that IDOL antisense (siIDOL) reduces IDOL mRNA expression in HepG2 cells. FIG. 3E illustrates rescue of human IDOL mRNA expression when miR-33a-3p and miR-33a-5p antisense inhibitors are present (left) and repression of LDLR expression in LXR-agonist (GW3965)-stimulated HepG2 in the presence of siRNA-mediated repression of IDOL with and without antisense-mediated inhibition by miR-33a-3p and miR-33a-5p (right). FIG. 3F illustrates increased LDLR expression in the presence of miR-33a-3p and miR-33a-5p expression in HepG2 cells and in the presence of siRNA-mediated IDOL inhibition (siIDOL). FIG. 3G shows human and mouse IDOL 3′-UTR sequences depicting miR-33a-3p and miR-33a-5p binding sites. The top sequences show IDOL sequences that can bind miR-33a-5p. The human IDOL 3′-UTR sequence at the top has the following sequence: AGAUGACCUUAUCGGGUGCAAUACUA (SEQ ID NO:26), while the top mouse IDOL 3′-UTR sequence is AGCUGACCUCAUCGGGUGCAAUACUA (SEQ ID NO: 27) with the miR-33a seed region highlighted. The miR-33a-5p sequence in the 5′ to 3′ direction has the following sequence GUGCAUUGUAGUUGCAUUGCA (SEQ ID NO: 25), with the seed region highlighted. The bottom sequences show IDOL sequences that can bind miR-33a-3p. The human IDOL 3′-UTR sequence at the bottom is UUCCACUCCCACUUGGGCAUUUUGGA (SEQ ID NO:28), while the mouse IDOL 3′-UTR sequence at the bottom is UUUCACCCCCACUUGGGCAUUUUGGA (SEQ ID NO:29). The human miR-33a-3p, has this 5′ to 3′ sequence: CAAUGUUUCCACAGUGCAUCAC (SEQ ID NO: 22, the active strand) with the seed region highlighted. FIG. 3H illustrates Ago2 PAR-CLIP analysis of mouse Bone Marrow Derived Macrophages (BMDM). A genome browser screenshot of the Mylip (IDOL) locus is shown with a density plot at the top showing RNA-seq data (RPKM, reads per kilobase per million mapped reads) from BMDM. PAR-CLIP RNA reads from Ago2-pulldown (blue bars) identified five major high-confidence miRNA binding sites. The Mylip (IDOL) transcript is schematically shown below the mRNAseq data, where the wider box to the left indicates the Mylip coding region, the narrower box indicates the Mylip 3′-UTR, and the arrows indicate the direction of transcription of the Mylip gene. Potential AGO2 binding sites are highlighted in red boxes. TargetScan 6.2 was used to identify miRNAs that interact with the miRNA binding sites identified by the Ago2 PAR-CLIP analysis. mRNA reads identity and miRNAs identified in this study are shown. RefSeq, reference sequence database. FIG. 3I graphically illustrates relative luciferase activity whose expression is driven by an operably linked wild type hsa-IDOL 3′UTR regulatory element (shown in FIG. 3G), or an operably linked mutant hsa-IDOL 3′UTR regulatory element, when miR-33a-3p, miR-33a-5p, or when a precursor control (PC) is used. As illustrated, miR-33a-3p and miR-33a-5p reduced luciferase expression from the wild type hsa-IDOL 3′UTR regulatory element, but mutation of the hsa-IDOL 3′UTR prevented such reduction of luciferase expression. FIG. 3J illustrates LDLR expression in IDOL-knockout Mouse Embryonic Fibroblasts (MEFs) in the presence of GW3965 (LXR-activation) or LPDS (Lipoprotein Deficient Serum) illustrating altered LDLR expression in response to miR-33-3p but not in response to miR-33-5p when IDOL is not present.

FIG. 4A-4L illustrate that high miR-33a-3p levels promote ABCA1 expression and cholesterol efflux. FIG. 4A shows that overexpression of miR-33a-3p increased ABCA1 protein expression in liver cells and human and mouse macrophages cell lines THP-1 and J774, respectively. MiR-33a-5p direct inhibition of ABCA1 served as a control. FIG. 4B shows that ABCA1 protein expression decreased at the indicated time points following addition of cycloheximide (CHX) to HepG2 cells to induce overexpression of miR-33a-3p in the presence of GW3965. FIG. 4C graphically illustrates the percent miR-33a-3p remaining at the indicated time points following addition of cycloheximide (CHX) in HepG2 cells to induce overexpression of miR-33a-3p in the presence of GW3965. FIG. 4D graphically illustrates quantification of ABCA1 mRNA by RT-PCR in mouse J774 cells overexpressing miR-33-3p or precursor control. FIG. 4E graphically illustrates expression of ABCA1 mRNA in HepG2 cells treated with indicated pre-microRNAs under normal (FBS), lipid depleted (LPDS) and LXR-activated (GW) conditions. FIG. 4F graphically illustrates cholesterol efflux from J774A.1 cells loaded with fluorescent-labeled cholesterol after transfection with miR-33-3p, miR-33-5p or control precursor (PC). FIG. 4G shows an immunoblot illustrating ABCA1 protein levels in the livers of HFD-fed mice injected with a liver-targeting miR-33a-3p mimic (n=5 per group) after short term (2 days) treatment. Control mice were injected with a mimic control (a scrambled microRNA). Actin was used as loading control. FIG. 4H graphically illustrates miR-33a-3p mimic levels in the liver of C57BL/6J mice injected with the miR-33a-3p mimic or a mimic control. FIG. 4I graphically illustrates ABCA1 protein levels as determined by densitometric analysis of actin normalized relative ABCA1 protein levels from a western blot of mouse live-injected with the miR-33a-3p mimic or a mimic control. Statistical significance between sets/groups was calculated by unpaired t-test, *P<0.05, **P<0.01, ***P<0.001. a.u., arbitrary units. For FIG. 4J-4L, 24-week-old high-carbohydrate diet (HCD)-fed mice were treated twice with a Locked Nucleic Acid (LNA) antisense miR-33-3p (LNA-anti-33a-3p) or control (Control LNA) over four days, and peritoneal macrophages were isolated from these mice for analysis. n=5 per group. FIG. 4J illustrates that short-term knockdown of miR-33-3p (using LNA-anti-33a-3p) in mice fed the HCD downregulates ABCA1 expression in macrophages analyzed by western blot. Actin was used as the loading control. The right panel shows the quantitation of ABCA1 expression. FIG. 4K graphically illustrates high-density lipoprotein (HDL)-cholesterol was reduced in the 24-week-old HCD-fed mice that were treated twice with the antisense miR-33-3p (LNA-anti-33a-3p) over four days compared to control (Control LNA). FIG. 4L illustrates that triglycerides were not reduced in the 24-week-old HCD-fed mice that were treated twice with the antisense miR-33-3p (LNA-anti-33a-3p) over four days compared to control (Control LNA).

FIG. 5A-5K illustrate that injection of a miR-33-3p mimic into C57BL/6J diet-induced obese (DIO) obese mice fed a high fat and cholesterol diet results in decreased serum PCSK9 and LDL levels and attenuated hepatic steatosis. FIG. 5A graphically illustrates expression of hepatic miR-33-3p and miR-33-5p as determined by quantitative RT-PCR in 42-weeks-old DIO mice injected with a liver-targeting miRNA control mimic or miR-33-3p mimic two times over the course of eight days. n=5 per group. FIG. 5B shows immunoblots illustrating expression levels of LDLR and PCSK9 in liver tissue from DIO mice treated with control or miR-33-3p mimic. Actin was used as a loading control. FIG. 5C graphically illustrates levels of circulating PCSK9 as measured by ELISA in the sera isolated from miR-33-3p mimic-treated DIO mice. FIG. 5D graphically illustrates reduced levels of LDL and HDL cholesterol in fractionated sera that had been isolated over time and pooled from miR-33-3p mimic-treated DIO mice compared to control mice treated with a mimic control. The fractions were analyzed by Fast Protein Liquid Chromatography (FPLC) (n=5). FIG. 5E graphically illustrates total cholesterol, LDL cholesterol and HDL cholesterol concentrations were reduced in sera from miR-33-3p mimic-treated DIO mice compared to control mice treated with a mimic control. FIG. 5F shows immunoblots and band intensities of ApoB 100 from isolated and pooled FPLC fractions of sera from DIO mice control or miR-33-3p mimic-treated DIO mice. FIG. 5G shows band intensities of the immunoblots of Apo A-I from isolated and pooled FPLC fractions of sera from DIO control or miR-33-3p mimic-treated mice. FIG. 5H shows an immunoblots of the indicated fractions illustrating Apo A-I from isolated and pooled FPLC fractions of mice sera from control or miR-33-3p mimic-treated DIO mice. Statistical significance between sets/groups was calculated by unpaired t-test, *P<0.05, **P<0.01, ***P<0.001. a.u., arbitrary units. FIG. 5I illustrates that increased VLDL secretion miR-33-3p mimic-treated mice was correlated with a marked decrease in hepatic lipid accumulation (oil red-stained), as shown by histological examination of livers from miR-33-3p mimic-treated mice compared to control mouse livers. FIG. 5J shows results from a longer study, illustrating that cholesterol levels in LDL and HDL were reduced in the miR-33a-3p mimic-treated mice compared to control-treated mice. FIG. 5K shows that PCSK9 and ANGPTL3 levels were reduced in the livers and sera of miR-33a-3p mimic-treated mice compared to control-treated mice in the longer study.

FIG. 6A-6H illustrate that short-term (two-day) exposure to the miR-33-3p mimic in diet-induced obese (DIO) mice fed a high-fat diet or a high carbohydrate diet consistently upregulates hepatic LDLR while lowering LDL-cholesterol, HDL-cholesterol, and LDL-associated triglycerides. FIG. 6A shows a western blot illustrating hepatic LDLR expression at various time points after high-fat diet mice received the miR-33-3p mimic or control. B-actin served as the loading control. FIG. 6B graphically illustrates levels of LDL and HDL-cholesterol isolated by fast protein liquid chromatography (FPLC) fractionation of pooled sera from the high-fat diet mice treated with control (upper dashed line) or miR-33-3p mimic (lower solid line) for 2 days. FIG. 6C graphically illustrates levels of VLDL-associated and LDL-associated triglycerides isolated by fast protein liquid chromatography (FPLC) fractionation of pooled sera from high-fat diet mice treated with control (square symbols, upper dashed line) or miR-33-3p mimic (circle symbols, lower solid line) for 2 days (n=5 per group). FIG. 6D-6F relate to short-term knockdown of miR-33-3p in high-carbohydrate diet (HCD)-fed mice. FIG. 6D illustrates hepatic expression of miR-33a-3p relative to miR-423-3p as determined by quantitative RT-PCR in 24-week-old high-carbohydrate diet (HCD)-fed mice treated twice with antisense LNA control (left bar) or LNA antisense miR-33-3p (right bar) over four days. FIG. 6E shows a western blot illustrating ABCA1 expression in peritoneal macrophages isolated from 24-week-old high-carbohydrate diet (HCD)-fed mice treated twice with antisense LNA control (left) or with LNA antisense miR-33-3p (right). Actin was used as the loading control. FIG. 6F graphically illustrates levels of ABCA1 expression relative to actin in 24-week-old high-carbohydrate diet (HCD)-fed mice treated twice with antisense LNA control (left bar) or LNA antisense miR-33-3p (right bar) over four days. FIG. 6G graphically illustrates levels of HDL-cholesterol in sera isolated from 24-week-old high-carbohydrate diet (HCD)-fed mice treated twice with antisense LNA control (circles, upper trace) or LNA antisense miR-33-3p (triangles, lower trace) over four days after separation of the sera by fast protein liquid chromatography (FPLC) fractionation. FIG. 6H graphically illustrates levels of VLDL-associated triglycerides in sera isolated from 24-week-old high-carbohydrate diet (HCD)-fed mice treated twice with antisense LNA control (circles, upper trace) or LNA antisense miR-33-3p (triangles, lower trace) over four days after separation of the sera by fast protein liquid chromatography (FPLC) fractionation.

FIG. 7A-7E illustrate the effects of once-per-week treatment with the miR-33a-3p mimic in CETP transgenic mice that express Cholesteryl Ester Transfer Protein in the liver and plasma when maintained on a high fat diet. FIG. 7A shows that once-per-week treatment with the miR-33a-3p mimic elevated plasma HDL (center), reduced plasma triglycerides (right), and reduced non-HDL-cholesterol in plasma (left). FIG. 7B shows that once-per-week treatment with the miR-33a-3p mimic promoted VLDL clearance. FIG. 7C shows that once-per-week treatment with the miR-33a-3p mimic reduced fat mass after 15 weeks without any significant alteration in food intake. FIG. 7D shows that once-per-week treatment with the miR-33a-3p mimic promoted fatty acid uptake by subcutaneous white adipose tissue (sWAT), reflecting increased LPL activity in the CETP transgenic mice. The uptake of fatty acids in visceral-gonadal white adipose tissues (GWAT), interscapular brown adipose tissues (iBAT), cervical-supraclavicular adipose tissue (sBAT), heart tissues, and spleen tissues is also shown. FIG. 7E shows that once-per-week treatment with the miR-33a-3p mimic prevented induced liver weight gain that can occur as a consequence of ER-stress and increased lipogenesis at postprandial state.

FIG. 8A-8D illustrate that miR-33a-3p mimic treatment improves LDL/HDL ratios in mice with non-alcoholic steatohepatitis (NASH) that were fed a high-fat/high-fructose/high cholesterol diet. FIG. 8A illustrates reduced cholesterol levels of VLDL, LDL, and HDL lipoproteins from NASH mice treated with the miR-33a-3p mimic or the non-treated control (mimic control). FIG. 8B shows immunoblots illustrating LDLR, ANGPTL3, and PCSK9 protein levels in NASH mice treated with the miR-33a-3p mimic or the non-treated control (MC, mimic control). FIG. 8C graphically illustrates relative RNA levels expressed by LDLR, PCSK9, ANGPTL3, ABCA1 genes of the miR-33a-3p mimic or the non-treated control (MC, mimic control) mice. FIG. 8D shows representative images of a wild type (untreated) mouse and a transgenic miR-33a-3p mouse, after both were kept on a 60% high fat diet for 12 weeks, showing that the liver of the transgenic miR-33a-3p mouse is healthy and red, indicating that it was protected from developing hepatic steatosis, a major event associated with NASH. n=3-5.

FIG. 9 is a schematic diagram illustrating some of the gene products and genes that miR-33a-3p mimics can modulate to increase LDL-uptake of fatty acids, to reduce LDL-associated cholesterol, and to increase postprandial VLDL processing.

### DETAILED DESCRIPTION

### Definitions

The terms “treat” and “treating” as used herein refer to (i) preventing a pathologic condition from occurring (e.g., prophylaxis); (ii) inhibiting the pathologic condition or arresting its development; (iii) relieving the pathologic condition; and/or (iv) ameliorating, alleviating, lessening, and removing symptoms of a condition. A compound, e.g., nucleic acid molecule, described herein may be in an amount in a formulation or medicament, which is an amount that can lead to a biological effect, or lead to ameliorating, alleviating, lessening, relieving, diminishing or removing symptoms of a condition, e.g., disease, for example.

The term “therapeutically effective amount” as used herein refers to an amount of a compound, or an amount of a combination of compounds, to treat, inhibit or prevent a disease or disorder, or to prevent, inhibit or treat a symptom of the disease or disorder, in a subject.

The terms “subject,” “patient” or “subject in need thereof” refers to a living organism suffering from or prone to a disease or condition that can be treated by administration of a compound, pharmaceutical composition, or mixture. Non-limiting examples include humans, other mammals, bovines, rats, mice, dogs, monkeys, goat, sheep, cows, deer, and other non-mammalian animals. In some embodiments, a patient is human. In some embodiments, a patient is a domesticated animal. In some embodiments, a patient is a dog. In some embodiments, a patient is livestock animal. In some embodiments, a patient is a mammal. In some embodiments, a patient is a cat. In some embodiments, a patient is a horse. In some embodiments, a patient is bovine. In some embodiments, a patient is a canine. In some embodiments, a patient is a feline. In some embodiments, a patient is a non-human primate. In some embodiments, a patient is a mouse. In some embodiments, a patient is a rat. In some embodiments, a patient is a newborn animal. In some embodiments, a patient is a newborn human. In some embodiments, a patient is a newborn mammal. In some embodiments, a patient is an elderly animal. In some embodiments, a patient is an elderly human. In some embodiments, a patient is an elderly mammal. In some embodiments, a patient is a geriatric patient.

As used herein, the term “isolated” in the context of nucleic acid molecule refers to a nucleic acid molecule which is separated from other molecules which are present in the natural source of the nucleic acid molecule.

As used herein, the terms “prevent”, “prevention” and “preventing” refer to obtaining a prophylactic benefit in a subject receiving a pharmaceutical composition. With respect to achieving a prophylactic benefit, the object is to delay or prevent the symptoms associated with the pathological condition or disorder. A “prophylactically effective amount” refers to that amount of a prophylactic agent, sufficient to achieve at least one prophylactic benefit in a subject receiving the composition.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

Polynucleotide modifications, e.g., for protecting exogenous polynucleotides from degradation, include, but are not limited to, addition of terminal amino group(s) and the use of modified internucleotide linkages such as, for example, phosphorothioates, phosphoramidates, and O-methyl ribose or deoxyribose residues.

Exemplary nucleic acid analogs may have a modified pyrimidine nucleobase, or a purine or pyrimidine base that contains an exocyclic amine.

Other nucleotide modifications include peptide nucleic acid (PNA) or locked nucleic acid (LNA), analogs of methyleneoxy (4′-CH₂-O-2′) BNA, phosphorothioate-methyleneoxy (4′-CH₂-O-2′) BNA and 2′-thio-BNAs, have also been prepared (Kumar et al., 1998), as well as amino- and 2′-methylamino-BNA. Preparation of locked nucleoside analogs comprising oligodeoxyribonucleotide duplexes as substrates for nucleic acid polymerases has also been described (WO 99/14226). Furthermore, synthesis of 2′-amino-BNA, a comformationally restricted high-affinity oligonucleotide analog has been described in the art (Singh et al., 1998).

Modified sugar moieties may be used, e.g., to alter, typically increase, the affinity of the polynucleotide for its target and/or increase nuclease resistance. A representative list of modified sugars includes but is not limited to bicyclic modified sugars (BNA's), including methyleneoxy (4′-CH₂-O-2′) BNA and ethyleneoxy (4′-(CH₂)₂-O-2′ bridge) BNA; substituted sugars, especially 2′-substituted sugars having a 2′-F, 2′—OCH₃ or a 2′-O(CH₂)₂—OCH₃ substituent group; and 4′-thio modified sugars. Sugars can also be replaced with sugar mimetic groups among others. Methods for the preparations of modified sugars are well known to those skilled in the art. Some representative patents and publications that teach the preparation of such modified sugars include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; 6,531,584; and 6,600,032; and WO 2005/121371.

Exemplary miRNA Sequences, Modifications and Compositions

In one embodiment, miRNA based nucleic acids useful in the methods are based on mature miRNA sequences, e.g., guide or active miRNAs that include but are not limited to 5′aauguuu3′ (SEQ ID NO:1), 5′caauguuuccacagugcaucac3′ (SEQ ID NO: 2), 5′aauguuuccacagugcaucac3′ (SEQ ID NO:3), 5′aauguuuccacagugcau3′ (SEQ ID NO: 4), 5′aauguuuccacagug3′ (SEQ ID NO:5), 5′aauguuuccaca3′ (SEQ ID NO:6), 5′caauguuuccacagugcaucac3′ (SEQ ID NO:7), 5′caauguuuccacagugcau3′ (SEQ ID NO: 8), 5′caauguuuccacagug3′ (SEQ ID NO:9), 5′caauguuuccaca3′ (SEQ ID NO:10), as well as the corresponding DNA sequences, and sequences having at least 90%, 92%, 95%, 96%, or 99% identity thereto. In one embodiment, the ribonucleotides or deoxyribonucleotides include one or more modified ribonucleotides or deoxyribonucleotides, e.g., modified phosphate linkages, modified sugars, modified nucleobases, or combinations thereof.

In one embodiment, miRNAs useful in the methods include but are not limited to 5′X1aauguuuX23′ (SEQ ID NO:11), wherein X1 and X2 are independently absent or are from 1 to 20 ribonucleotides in length, e.g., X1 or X2 are 1, 2, 3, 4, 5, 6, 7, 8, 9, 10, or 15 ribonucleotides in length, as well as the corresponding DNA sequences, and sequences having at least 90%, 92%, 95%, 96%, or 99% identity thereto. In one embodiment, the ribonucleotides or deoxyribonucleotides include one or more modified ribonucleotides or deoxyribonucleotides, e.g., modified phosphate linkages, modified sugars, modified nucleobases, or combinations thereof.

In one embodiment, miRNAs useful in the methods include but are not limited to 5′X1X3auguuuX23′ (SEQ ID NO: 12), wherein X3 is not a, and wherein X1 and X2 are independently absent or are from 1 to 20 ribonucleotides in length, e.g., X1 or X2 are 1, 2, 3, 4, 5, 6, 7, 8, 9, 10, or 15 ribonucleotides in length, as well as the corresponding miRNA DNA sequences, and sequences having at least 90%, 92%, 95%, 96%, or 99% identity thereto. In one embodiment, the ribonucleotides or deoxyribonucleotides include one or more modified ribonucleotides or deoxyribonucleotides, e.g., modified phosphate linkages, modified sugars, modified nucleobases, or combinations thereof.

In one embodiment, miRNAs useful in the methods include but are not limited to 5′X1aX3uguuuX23′ (SEQ ID NO:13), wherein X3 is not a, and X1 and X2 are independently absent or are from 1 to 20 ribonucleotides in length, e.g., X1 or X2 are 1, 2, 3, 4, 5, 6, 7, 8, 9, 10, or 15 ribonucleotides in length, as well as the corresponding miRNA DNA sequences, and sequences having at least 90%, 92%, 95%, 96%, or 99% identity thereto. In one embodiment, the ribonucleotides or deoxyribonucleotides include one or more modified ribonucleotides or deoxyribonucleotides, e.g., modified phosphate linkages, modified sugars, modified nucleobases, or combinations thereof.

In one embodiment, miRNAs useful in the methods include but are not limited to 5′X1aaX3guuuX23′ (SEQ ID NO:14), wherein X3 is not u, and X1 and X2 are independently absent or are from 1 to 20 ribonucleotides in length, e.g., X1 or X2 are 1, 2, 3, 4, 5, 6, 7, 8, 9, 10, or 15 ribonucleotides in length, as well as the corresponding DNA sequences, and sequences having at least 90%, 92%, 95%, 96%, or 99% identity thereto. In one embodiment, the ribonucleotides or deoxyribonucleotides include one or more modified ribonucleotides or deoxyribonucleotides, e.g., modified phosphate linkages, modified sugars, modified nucleobases, or combinations thereof.

In one embodiment, miRNAs useful in the methods include but are not limited to 5′X1aauX3uuuX23′ (SEQ ID NO:15), wherein X3 is not g, and X1 and X2 are independently absent or are from 1 to 20 ribonucleotides in length, e.g., X1 or X2 are 1, 2, 3, 4, 5, 6, 7, 8, 9, 10, or 15 ribonucleotides in length, as well as the corresponding DNA sequences, and sequences having at least 90%, 92%, 95%, 96%, or 99% identity thereto. In one embodiment, the ribonucleotides or deoxyribonucleotides include one or more modified ribonucleotides or deoxyribonucleotides, e.g., modified phosphate linkages, modified sugars, modified nucleobases, or combinations thereof.

In one embodiment, miRNAs useful in the methods include but are not limited to 5′X1aaugX3uuX23′ (SEQ ID NO:16), wherein X3 is not u, and X1 and X2 are independently absent or are from 1 to 20 ribonucleotides in length, e.g., X1 or X2 are 1, 2, 3, 4, 5, 6, 7, 8, 9, 10, or 15 ribonucleotides in length, as well as the corresponding miRNA DNA sequences, and sequences having at least 90%, 92%, 95%, 96%, or 99% identity thereto. In one embodiment, the ribonucleotides or deoxyribonucleotides include one or more modified ribonucleotides or deoxyribonucleotides, e.g., modified phosphate linkages, modified sugars, modified nucleobases, or combinations thereof.

In one embodiment, miRNAs useful in the methods include but are not limited to 5′X1aauguX3uX23′ (SEQ ID NO:17), wherein X3 is not u, and X1 and X2 are independently absent or are from 1 to 20 ribonucleotides in length, e.g., X1 or X2 are 1, 2, 3, 4, 5, 6, 7, 8, 9, 10, or 15 ribonucleotides in length, as well as the corresponding DNA sequences, and sequences having at least 90%, 92%, 95%, 96%, or 99% identity thereto. In one embodiment, the ribonucleotides or deoxyribonucleotides include one or more modified ribonucleotides or deoxyribonucleotides, e.g., modified phosphate linkages, modified sugars, modified nucleobases, or combinations thereof.

In one embodiment, miRNAs useful in the methods include but are not limited to 5′X1aauguuX3X23′ (SEQ ID NO:18), wherein X3 is not u, and X1 and X2 are independently absent or are from 1 to 20 ribonucleotides in length, e.g., X1 or X2 are 1, 2, 3, 4, 5, 6, 7, 8, 9, 10, or 15 ribonucleotides in length, as well as the corresponding m DNA sequences. In one embodiment, the ribonucleotides or deoxyribonucleotides include one or more modified ribonucleotides or deoxyribonucleotides, e.g., modified phosphate linkages, modified sugars, modified nucleobases, or combinations thereof.

Additional examples of miR-33 sequences from a variety of species are shown in Table 1 below, with the seed sequence highlighted in bold and with underlining. As illustrated, the seed sequence is highly conserved between species.

TABLE 1
miR-33 Sequences from Various Species

| Species | Sequence | SEQ ID NO: |
|---|---|---|
| hsa-miR-33a-3p (humans | caauguuuccacagugcaucac | SEQ ID NO: 30 |
| and non-human primates) | | |
| mmu-miR-33-3p (mouse) | caauguuuccacagugcaucac | SEQ ID NO: 31 |
| ocu-miR-33a-3p | caauguuuccacagugcaucc | SEQ ID NO: 32 |
| (European rabbit) | | |
| oha-miR-33a-3p | caauguuucugcagugcagu | SEQ ID NO: 33 |
| (King cobra) | | |
| cpo-miR-33a-3p | caauucguuuccacagugcauca | SEQ ID NO: 34 |
| (Guinea pig) | | |
| chi-miR-33a-3p (Goat) | caauguuuccacagugcaa | SEQ ID NO: 35 |
| dno-miR-33a-3p (Nine- | caauguguccacagugcaucc | SEQ ID NO: 36 |
| banded armadillo) | | |
| oni-miR-33a-3p | caaugugucugcagugcagua | SEQ ID NO: 37 |
| (Nile tilapia) | | |
| ssa-miR-33a-3p | caaugugucugcagugcagua | SEQ ID NO: 38 |
| (Atlantic salmon) | | |
| xla-miR-33a-3p | caaugugccugcagugcaaca | SEQ ID NO: 39 |
| (African clawed frog) | | |
| pal-miR-33a-3p | caguguuuccacagugcauca | SEQ ID NO: 40 |
| (Black flying fox) | | |
| oan-miR-33a-3p | caaugccccugcagugcaau | SEQ ID NO: 41 |
| (Platypus) | | |

In one embodiment, miRNAs useful in the methods include but are not limited to any including one or more of SEQ ID NOs: 30-41, well as the corresponding DNA 5 sequences, and sequences having at least 90%, 92%, 95%, 96%, or 99% identity thereto. Such miRNAs can be from 1 to 20 ribonucleotides in length. In one embodiment, the ribonucleotides or deoxyribonucleotides include one or more modified ribonucleotides or deoxyribonucleotides, e.g., modified phosphate linkages, modified sugars, modified nucleobases, or combinations thereof.

In one embodiment, one or more types of miRNAs are in the form of a double-stranded or triple stranded molecule. For example, an antisense sequence (passenger strand) of any of the molecules described above may be employed to form a double stranded molecule, e.g., in a hairpin-loop structure or two separate strands. In one embodiment, the modifications in ribonucleotides or deoxyribonucleotide are in the antisense strand. In one embodiment, the modifications in the modified ribonucleotides or deoxyribonucleotide are in the sense strand. In one embodiment, the modifications are not in the seed region. In one embodiment, a modification is a Locked Nucleic Acid (LNA), also known as bridged nucleic acid (BNA), and often referred to as inaccessible RNA. Such LNA ribonucleotides or deoxyribonucleotide have a modified nucleotide in which the ribose moiety is modified with an extra bridge connecting the 2′ oxygen and 4′ carbon. In one embodiment, the modification includes one or more phosphorothioate groups, modification at 2-hydroxyl groups in sugar, modifications that enhance stability, e.g., decrease degradation rates of the sense strand, or decrease stability, e.g., of the antisense strand after it is dissociated from the sense strand. In one embodiment, the antisense strand may be chemically coupled to a molecule that enhances uptake, e.g., associated with or chemically coupled to cholesterol or a lipid.

The nucleic acid molecules, sense or antisense, may be of any length. In one embodiment, the sense nucleic acid molecule may be from 6 to 100 nucleotides in length, e.g., from 6 to 22, 6 to 25, 6 to 30, 20 to 30, 30 to 40, or 50 to 100 nucleotides in length. In one embodiment, the antisense nucleic acid molecule may be from 6 to 100 nucleotides in length, e.g., from 6 to 22, 6 to 25, 6 to 30, 20 to 30, 30 to 40, or 50 to 100 nucleotides in length. In one embodiment, the sense nucleic acid molecule is shorter than the antisense nucleic acid molecule. In one embodiment, for single stranded nucleic acid molecules that form hairpin-loops, the nucleic acid molecule may be from 14 to 200 nucleotides in length, e.g., from 14 to 25, 14 to 30, 20 to 40, 50 to 100, or 100 to 200 nucleotides in length.

### Exemplary Liver Targeting Moieties

In one embodiment, formulations having liver targeting moieties may be recognized selectively by liver cells, e.g., receptors present on liver cells such as asialoglycoprotein receptor. The targeting moiety may compete with an endogenously produced ligand. The targeting formulation may be nontoxic, biocompatible, biodegradable, and/or physico-chemically stable in vivo. The formulation may have uniform sinusoid capillary distribution, and/or controllable and predictable rate of release of the miRNA or corresponding DNA. In one embodiment, formulations having liver targeting moieties may cross the anatomical barriers such as those of stomach and intestine and minimize drug leakage during its passage through stomach, intestine, and other parts of the body.

For liver targeting, the formulation may include one or more of galactose, lactose, galactosamine, RGD, lacto bionic bcid (LA) ligand, lactoferrin, soybean-derived SG ligand, bile acid, mannose, glycyrrhizin, glycyrrhetinic acid, Hepatitis B antigen, multiantennary N-glycans, complex-type desialylated glycans, such as asialofetuin A (desialylated alpha-2-HS-glycoprotein) or asialoorosomucoid (desialylated alpha-1-acid-glycoprotein or other desialylated glycans with terminal galactose (Gal) or N-acetyl galactosamine (GalNAc) residues, or a molecule that binds to asialoglycoprotein (ASGP)-receptor, transferrin receptor, HDL-R, LDL-R, IgA-R, or scavenger receptor. In one embodiment the formulation, e.g., cationic liposome, comprises a liver cell-specific binding ligand that allows for endocytosis of, for example, a liposome having nucleic acid comprising miRNA-33a-3p. In one embodiment the formulation, e.g., cationic liposome, comprises an antibody that binds liver cells and allows for endocytosis of the formulation.

### Exemplary Delivery Vehicles

The nucleic acid described herein may be delivered by any of a variety of vehicles including but not limited to viruses, liposomes, or other nanoparticles. The nucleic acid may form complexes with one or more non-nucleic acid molecules or may be encapsulated in or on the surface of delivery vehicles such as nanoparticles.

Numerous lipids which are used in liposome delivery systems may be used to form a lipid layer, e.g., a bilayer. Exemplary lipids for use include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine](DOPS), 1,2-dioleoyl-3-trimethylammonium-propane (18:1 DOTAP), 1,2-dioleoyl-sn-glycero-3-phospho-(1′-ruc-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy (polyethylene glycol)-2000](18:1 PEG-2000 PE), 1,2-dipalmitoyl-sn-glycero-3-phosphoethanolamine-N-[methoxy (polyethylene glycol)-2000](16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. Cholesterol, not technically a lipid, but presented as a lipid for purposes of an embodiment. Often cholesterol is incorporated into lipid bi-layers to enhance structural integrity of the bi-layer. DOPE and DPPE may be particularly useful for conjugating (through an appropriate crosslinker) a targeting moiety, e.g., a liver targeting moiety on the lipid.

In one embodiment, anionic liposomal nanoparticles are employed as a delivery vehicle for the nucleic acid molecules, wherein the anionic liposomal nanoparticles optionally comprise one or more targeting moieties. In one embodiment, the anionic liposomal nanoparticles have diameters of about 100 nm to about 500 nm. In one embodiment, the anionic liposomal nanoparticles have diameters of about 150 nm to about 250 nm. In one embodiment, the lipid layer comprises lipids including but not limited to 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(l′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy (polyethylene glycol)-2000] (18:1 PEG-2000 PE), 1,2-dipalmitoyl-sn-glycero-3-phosphoethanolamine-N-[methoxy (polyethylene glycol)-2000] (16:0 PEG-2000 PE), 1-oleoyl-2-[12-[(7-nitro-2-1,3-benzoxadiazol-4-yl)amino] lauroyl]-sn-glycero-3-phosphocholine (18:1-12:0 NBD PC), 1-palmitoyl-2-{12-[(7-nitro-2-1,3-benzoxadiazol-4-yl)amino] lauroyl}-sn-glycero-3-phosphocholine (16:0-12:0 NBD PC), and mixtures thereof; or wherein said lipid layer comprises 1,2-distearoyl-sn-glycero-3-phosphocholine (DSPC), 1,2-dioleoyl-sn-glycero-3-phosphoethanolamine (DOPE), or a mixture thereof; or wherein said lipid layer comprises cholesterol. In one embodiment, the lipid layer comprises two or more of DPPC, DMPG or cholesterol.

In certain embodiments, liposomes generally range in size from about 8 to 10 nm to about 5 μm in diameter, e.g., about 20-nm to 3 μm in diameter, about 10 nm to about 500 nm, about 20-200-nm (including about 150 nm, which may be a mean or median diameter), about 50 nm to about 150 nm, about 75 to about 130 nm, or about 75 to about 100 nm as well as about 200 to about 450 nm, about 100 to about 200 nm, about 150 to about 250 nm, or about 200 to about 300 nm.

In certain embodiments, the delivery vehicle may be a biodegradable polymer comprising one or more aliphatic polyesters, poly (lactic acid) (PLA), poly (glycolic acid) (PGA), co-polymers of lactic acid and glycolic acid (PLGA), polycaprolactone (PCL), polyanhydrides, poly (ortho) esters, polyurethanes, poly (butyric acid), poly (valeric acid), poly (lactide-co-caprolactone), alginate and other polysaccharides, collagen, and chemical derivatives thereof, albumin a hydrophilic protein, zein, a prolamine, a hydrophobic protein, and copolymers and mixtures thereof.

In other embodiments, the lipid bi-layer is comprised of a mixture of DSPC, DOPC and optionally one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), a lipid mixture comprising (in molar percent) between about 50% to about 70% or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-choline, DMPC [14:0] having a carbon length of 14 and no unsaturated bonds, 1,2-dipalmitoyl-sn-glycero-3-phosphocholine (DPPC) [16:0], POPC [16:0-18:1] and DOTAP [18:1]; and wherein (b) the molar concentration of DSPC and DOPC in the mixture is between about 10% to about 99% or about 50% to about 99%, or about 12% to about 98%, or about 13% to about 97%, or about 14% to about 96%, or about 55% to about 95%, or about 56% to about 94%, or about 57% to about 93%, or about 58% to about 42%, or about 59% to about 91%, or about 50% to about 90%, or about 51% to about 89%.

In certain embodiments, the lipid bi-layer is comprised of one or more compositions selected from the group consisting of a phospholipid, a phosphatidylcholine, a phosphatidyl-serine, a phosphatidyl-diethanolamine, a phosphatidylinosite, a sphingolipid, and an ethoxylated sterol, or mixtures thereof. In illustrative examples of such embodiments, the phospholipid can be a lecithin; the phosphatidylinosite can be derived from soy, rape, cotton seed, egg and mixtures thereof; the sphingolipid can be ceramide, a cerebroside, a sphingosine, and a sphingomyelin, and a mixture thereof; the ethoxylated sterol can be phytosterol, PEG-(polyethyleneglycol)-5-soy bean sterol, and PEG-(polyethyleneglycol)-5 rapeseed sterol. In certain embodiments, the phytosterol comprises a mixture of at least two of the following compositions: sitosterol, campesterol and stigmasterol.

In still other illustrative embodiments, the lipid bi-layer is comprised of one or more phosphatidyl groups selected from the group consisting of phosphatidyl choline, phosphatidyl-ethanolamine, phosphatidyl-serine, phosphatidyl-inositol, lyso-phosphatidyl-choline, lyso-phosphatidyl-ethanolamine, lyso-phosphatidyl-inositol and lyso-phosphatidyl-inositol.

In still other illustrative embodiments, the lipid bi-layer is comprised of phospholipid selected from a monoacyl or diacylphosphoglyceride.

In still other illustrative embodiments, the lipid bi-layer is comprised of one or more phosphoinositides selected from the group consisting of phosphatidyl-inositol-3-phosphate (PI-3-P), phosphatidyl-inositol-4-phosphate (PI-4-P), phosphatidyl-inositol-5-phosphate (PI-5-P), phosphatidyl-inositol-3,4-diphosphate (PI-3,4-P2), phosphatidyl-inositol-3,5-diphosphate (PI-3,5-P2), phosphatidyl-inositol-4,5-diphosphate (PI-4,5-P2), phosphatidyl-inositol-3,4,5-triphosphate (PI-3,4,5-P3), lysophosphatidyl-inositol-3-phosphate (LPI-3-P), lysophosphatidyl-inositol-4-phosphate (LPI-4-P), lysophosphatidyl-inositol-5-phosphate (LPI-5-P), lysophosphatidyl-inositol-3,4-diphosphate (LPI-3,4-P2), lysophosphatidyl-inositol-3,5-diphosphate (LPI-3,5-P2), lysophosphatidyl-inositol-4,5-diphosphate (LPI-4,5-P2), and lysophosphatidyl-inositol-3,4,5-triphosphate (LPI-3,4,5-P3), and phosphatidyl-inositol (PI), and lysophosphatidyl-inositol (LPI).

In still other illustrative embodiments, the lipid bi-layer is comprised of one or more phospholipids selected from the group consisting of PEG-poly (ethylene glycol)-derivatized distearoylphosphatidylethanolamine (PEG-DSPE), PEG-poly (ethylene glycol)-derivatized dioleoylphosphatidylethanolamine (PEG-DOPE), poly (ethylene glycol)-derivatized ceramides (PEG-CER), hydrogenated soy phosphatidylcholine (HSPC), egg phosphatidylcholine (EPC), phosphatidyl ethanolamine (PE), phosphatidyl glycerol (PG), phosphatidyl inositol (PI), monosialoganglioside, sphingomyelin (SPM), distearoylphosphatidylcholine (DSPC), dimyristoylphosphatidylcholine (DMPC), and dimyristoylphosphatidylglycerol (DMPG).

In still other embodiments, the lipid bi-layer comprises one or more PEG-containing phospholipids, for example 1,2-dioleoyl-sn-glycero-3-phosphoethanolamine-N-[methoxy (polyethylene glycol)] (ammonium salt) (DOPE-PEG), 1,2-distearoyl-sn-glycero-3-phosphoethanolamine-N-[methoxy (polyethylene glycol)] (ammonium salt) (DSPE-PEG), 1,2-distearoyl-sn-glycero-3-phosphoethanolamine-N-[amino (polyethylene glycol)] (DSPE-PEG-NH₂) (DSPE-PEG). In the PEG-containing phospholipid, the PEG group ranges from about 2 to about 250 ethylene glycol units, about 5 to about 100, about 10 to 75, or about 40-50 ethylene glycol units. In certain exemplary embodiments, the PEG-phospholipid is 1,2-dioleoyl-sn-glycero-3-phosphoethanolamine-N-[methoxy (polyethylene glycol)-2000] (ammonium salt) (DOPE-PEG₂₀₀₀), 1,2-distearoyl-sn-glycero-3-phosphoethanolamine-N-[methoxy (polyethylene glycol)-2000] (ammonium salt) (DSPE-PEG₂₀₀₀), 1,2-distearoyl-sn-glycero-3-phosphoethanolamine-N-[amino (polyethylene glycol)-2000] (DSPE-PEG₂₀₀₀-NH₂) which can be used to covalent bind a functional moiety to the lipid bi-layer.

In certain embodiments, the lipid bi-layer is comprised of one or more phosphatidylcholines (PCs) selected from the group consisting of 1,2-distearoyl-sn-glycero-3-phosphocholine (DSPC) [18:0], 1,2-dioleoyl-sn-glycero-3-phosphocholine (DOPC) [18:1 (49-Cis)], 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), egg PC, and a lipid mixture comprising of one or more unsaturated phosphatidyl-cholines, DMPC [14:0] having a carbon length of 14 and no unsaturated bonds, 1,2-dipalmitoyl-sn-glycero-3-phosphocholine (DPPC) [16:0], POPC [16:0-18:1], and DOTAP [18:1]. The use of DSPC and/or DOPC as well as other zwitterionic phospholipids as a principal component (often in combination with a minor amount of cholesterol) is employed in certain embodiments in order to provide a protocell with a surface zeta potential which is neutral or close to neutral in character.

Cationic liposomes may be formed from a single type of lipid, or a combination of two or more distinct lipids. For instance, one combination may include a cationic lipid and a neutral lipid, or a cationic lipid and a non-cationic lipid. Exemplary lipids for use in the cationic liposomes include but are not limited to DOTAP, DODAP, DDAB, DOTMA, MVL5, DPPC, DSPC, DOPE, DPOC, POPC, or any combination thereof. In one embodiment, the cationic liposome has one or more of the following lipids or precursors thereof: N-[1-(2,3-dioleyloxy) propyl]-N,N,N-trimethylammonium chloride with a monovalent cationic head; N,N-dioctadecyl-N-4,8-diaza-10-aminodecanoyl glycine amide; 1,4,7,10-tetraazacyclododecane cyclen; imidazolium-containing cationic lipid having different hydrophobic regions (e.g., cholesterol and diosgenin); 1,2-dioleoyl-sn-glycero-3-phosphoethanolamine (DOPE); 3β-[N-(N′,N′-dimethylamino-ethane) carbamoyl) cholesterol (DC-Chol) and DOPE; O,O′-ditetradecanoyl-N-(«-trimethyl ammonioacetyl) diethanol-amine chloride, DOPE and cholesterol, phosphatidylcholine; 1,2-dilinoleyl-4-(2-dimethylaminoethyl)-[1,3]-dioxolane, 1,2-distearoyl-sn-glycerol-3-phosphocholine (DSPC) and cholesterol, 1-palmitoyl-2-oleoyl-sn-glycero-3-phosphocholine, DOPE, and 1,2-distearoyl-sn-glycero-3-phosphoethanolamine-N-(methoxy [polyethylene glycol-2000), 1,2-di-O-octadecenyl-3-trimethylammonium propane, cholesterol, and D-a-toco; 1,2-dioleoyl-3-trimethylammonium-propane, cholesterol; 3-β(N-(N′,N′-dimethyl, N′-hydroxyethyl amino-propane) carbamoyl) cholesterol iodide, DMHAPC-Chol and DOPE in equimolar proportion, or 1-palmitoyl-2-oleoyl-sn-glycero-3-ethylphosphocholine: cholesterol, dimethyldioctadecylammonium (DDAB); 1,2-di-O-octadecenyl-3-trimethylammonium propane; N1-[2-((1S)-1-{(3-aminopropyl)amino]-4-[di (3-amino-propyl)amino)amino] butylcarboxamido)ethyl]-3,4-di [oleyloxy]-benzamide (MVL5); 1,2-dioleoyl-3-dimethylammonium-propane (DODAP); 1,2-di-O-octadecenyl-3-trimethylammonium propane (DOTMA); 1,2-dioleoyl-sn-glycero-3-phosphocholine (DOPC); 1-palmitoyl-2-oleoyl-sn-glycero-3-phosphocholine (POPC).

### Routes and Formulations

Administration of compositions having one or more nucleic acid molecules disclosed herein, can be via any of suitable route of administration, particularly parenterally, for example, intravenously, intra-arterially, intraperitoneally, intrathecally, intraventricularly, intracranially, intramuscularly, or subcutaneously. Such administration may be as a single bolus injection, multiple injections, or as a short- or long-duration infusion. Implantable devices (e.g., implantable infusion pumps) may also be employed for the periodic parenteral delivery over time of equivalent or varying dosages of the particular formulation. For such parenteral administration, the nucleic acid compounds may be formulated as a sterile solution in water or another suitable solvent or mixture of solvents. The solution may contain other substances such as salts, sugars (particularly glucose or mannitol), to make the solution isotonic with blood, buffering agents such as acetic, citric, and/or phosphoric acids and their sodium salts, and preservatives.

The compositions alone or in combination with other active agents can be formulated as pharmaceutical compositions and administered to a mammalian host, such as a human patient in a variety of forms adapted to the chosen route of administration, e.g., orally or parenterally, by intravenous, intramuscular, topical or subcutaneous routes.

Thus, the compositions alone or in combination with another active agent, may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the composition having nucleic acid, optionally in combination with another active compound, may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of the nucleic acid and optionally other active compound in such useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the composition optionally in combination with another active compound may be incorporated into sustained-release preparations and devices.

The composition having nucleic acid optionally in combination with another active compound may also be administered intravenously or intraperitoneally by infusion or injection. Solutions of the nucleic acid molecule optionally in combination with another active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the nucleic acid which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of particle size in the case of dispersions or by the use of surfactants. The prevention of the action of microorganisms during storage can be brought about by various antibacterial and antifungal agents, for example, parabens, chlorobutanol, phenol, sorbic acid, thimerosal, and the like. In many cases, it may be useful to include isotonic agents, for example, sugars, buffers or sodium chloride. Prolonged absorption of the injectable compositions can be brought about by the use in the compositions of agents delaying absorption, for example, aluminum monostearate and gelatin, or a combination thereof.

For example, sterile injectable solutions are prepared by incorporating compound(s) in an effective amount in the appropriate solvent with various of the other ingredients enumerated above, followed by filter sterilization. Generally, dispersions can be prepared by incorporating the selected sterilized active ingredient(s), e.g., via filer sterilization, into a sterile vehicle that contains the basic dispersion medium and any other optional ingredients from those enumerated above. The compositions disclosed herein may also be formulated in a neutral or salt form. Upon formulation, solutions will be administered in a manner compatible with the dosage formulation, and in such amount as is effective for the intended application. The formulations are readily administered in a variety of dosage forms such as injectable solutions, topical preparations, oral formulations, including sustain-release capsules, hydrogels, colloids, viscous gels, transdermal reagents, intranasal and inhalation formulations, and the like. For administration of an injectable aqueous solution, without limitation, the solution may be suitably buffered, if necessary, and the liquid diluent first rendered isotonic with sufficient saline or glucose. These particular aqueous solutions are especially suitable for intravenous, intramuscular, subcutaneous, transdermal, subdermal, and/or intraperitoneal administration. In this regard, the compositions of the present disclosure may be formulated in one or more pharmaceutically acceptable vehicles, including for example sterile aqueous media, buffers, diluents, and the like. For example, a given dosage of active ingredient(s) may be dissolved in a particular volume of an isotonic solution (e.g., an isotonic NaCl-based solution), and then injected at the proposed site of administration, or further diluted in a vehicle suitable for intravenous infusion (see, e.g., “REMINGTON'S PHARMACEUTICAL SCIENCES” 15th Ed., pp. 1035-1038 and 1570-1580). While some variation in dosage will necessarily occur depending on the condition of the subject being treated, the extent of the treatment, and the site of administration, the person responsible for administration will nevertheless be able to determine the correct dosing regimens appropriate for the individual subject using ordinary knowledge in the medical and pharmaceutical arts.

In the case of sterile powders for the preparation of sterile injectable solutions, one method of preparation includes vacuum drying and the freeze drying techniques, which yield a powder of the active ingredient plus any additional desired ingredient present in the previously sterile-filtered solutions.

For topical administration, the composition optionally in combination with another active compound may be applied in pure form, e.g., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and antimicrobial agents can be added to optimize the properties for a given use.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

In addition, in one embodiment, the invention provides various dosage formulations of the nucleic acid optionally in combination with another active compound for inhalation delivery. For example, formulations may be designed for aerosol use in devices such as metered-dose inhalers, dry powder inhalers and nebulizers.

Useful dosages can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the nucleic acid optionally in combination with another active compound in a liquid, solid or gel composition, may be from about 0.1-25 wt-%, e.g., from about 0.5-10 wt-%, from 10 to 30 wt-%, 30 to 50-wt %, 50 to 70-wt %, or about 70 to 90 wt-%. The concentration in a semi-solid or solid composition such as a gel or a powder may be about 0.1-5 wt-%, e.g., about 0.5-2.5 wt-% or about 0.5-10 wt-%, from 10 to 30 wt-%, 30 to 50-wt %, 50 to 70-wt %, or about 70 to 90 wt-%.

The active ingredient may be administered to achieve peak plasma concentrations of the active compound of from about 0.5 to about 75 μM, e.g., about 1 to 50 μM, such as about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The amount of the nucleic acid optionally in combination with another active compound, or an active salt or derivative thereof, for use in treatment will vary not only with the particular salt selected but also with the route of administration, the nature of the condition being treated and the age and condition of the patient and will be ultimately at the discretion of the attendant physician or clinician. In general, however, a suitable dose may be in the range of from about 0.5 to about 100 mg/kg, e.g., from about 10 to about 75 mg/kg of body weight per day, such as 3 to about 50 mg per kilogram body weight of the recipient per day, for instance in the range of 6 to 90 mg/kg/day, e.g., in the range of 15 to 60 mg/kg/day. In one embodiment, 1 mg/kg to 100 mg/kg, e.g., per day, is administered. In one embodiment, 1 mg/kg to 20 mg/kg, e.g., per day, is administered. In one embodiment, 20 mg/kg to 40 mg/kg, e.g., per day, is administered. In one embodiment, 40 mg/kg to 60 mg/kg, e.g., per day, is administered. In one embodiment, 60 mg/kg to 80 mg/kg, e.g., per day, is administered. In one embodiment, 80 mg/kg to 100 mg/kg, e.g., per day, is administered. The nucleic acid optionally in combination with another active compound may be conveniently administered in unit dosage form; for example, containing 5 to 1000 mg, conveniently 10 to 750 mg, most conveniently, 50 to 500 mg of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye. The dose, and perhaps the dose frequency, will also vary according to the age, body weight, condition, and response of the individual patient. In general, the total daily dose range for an active agent for the conditions described herein, may be from about 50 mg to about 5000 mg, in single or divided doses. In one embodiment, a daily dose range should be about 100 mg to about 4000 mg, e.g., about 1000-3000 mg, in single or divided doses, e.g., 750 mg every 6 hr of orally administered compound. This may achieve plasma levels of about 500-750 μM, In managing the patient, the therapy should be initiated at a lower dose and increased depending on the patient's global response.

The amount, dosage regimen, formulation, and administration of nucleic acid disclosed herein will be within the purview of the ordinary-skilled artisan having benefit of the present teaching. It is likely, however, that the administration of a therapeutically-effective amount of the disclosed compositions may be achieved by multiple, or successive administrations, over relatively short or even relatively prolonged periods, as may be determined by the medical practitioner overseeing the administration of such compositions to the selected individual. However, a single administration, such as, without limitation, a single injection of a sufficient quantity of the delivered agent may provide the desired benefit to the patient for a period of time.

In certain embodiments, the present disclosure concerns formulation of one or more cationic nanoparticles, e.g., cationic liposomes, for administration to an animal. In one embodiment, a cationic liposome comprises two or more distinct lipids, one of the lipids is cationic, e.g., DOTAP is a cationic lipid, and at least one of the others is non-cationic, e.g., DPPC or DSPC. Ratios of the two or more distinct lipids can vary, for example, for two distinct lipids, the ratio of a non-cationic lipid, e.g., neutral lipid, to the cationic lipid may be x: 1 wherein x>1, x=1 or x: 1 where x<1. In one embodiment, x>1. The formulation of pharmaceutically acceptable excipients and carrier solutions is well known to those of ordinary skill in the art, as is the development of suitable dosing and treatment regimens for using the particular cationic nanoparticle compositions described herein in a variety of treatment regimens. In certain circumstances it will be desirable to deliver the disclosed compositions in suitably-formulated pharmaceutical vehicles by one or more standard delivery methods, including, without limitation, subcutaneously, parenterally, intravenously, intramuscularly, intrathecally, orally, intraperitoneally, transdermally, topically, by oral or nasal inhalation, or by direct injection to one or more cells, tissues, or organs within or about the body of an animal. The methods of administration may also include those modalities as described in U.S. Pat. Nos. 5,543,158; 5,641,515, and 5,399,363, each of which is specifically incorporated herein in its entirety by express reference thereto. Solutions of the active compounds as freebase or pharmacologically acceptable salts may be prepared in sterile water, and may be suitably mixed with one or more surfactants, such as hydroxypropylcellulose. Dispersions may also be prepared in glycerol, liquid polyethylene glycols, oils, or mixtures thereof. Under ordinary conditions of storage and use, these preparations contain a preservative to prevent the growth of microorganisms.

### EXEMPLARY EMBODIMENTS

In one embodiment, a method to prevent, inhibit or treat liver or cardiovascular disease in a mammal is provided. In one embodiment, a method to prevent, inhibit or treat liver disease in a mammal is provided. The method includes administering to a mammal in need thereof an effective amount of a composition comprising a nucleic acid sequence comprising a seed region of miRNA-33a-3p. In one embodiment, the mammal is a human. In one embodiment, the disease is steatosis, non-alcoholic fatty liver disease (NAFLD), or nonalcoholic steatohepatitis (NASH). In one embodiment, the mammal has alcohol fatty liver disease or chronic liver disease. In one embodiment, the mammal has atherosclerosis or complications thereof. In one embodiment, the mammal has hyperlipidemia or complications thereof. In one embodiment, the mammal has dyslipidemia or complications thereof. In one embodiment, the mammal has hypercholesteremia or complications thereof. In one embodiment, the composition comprises liposomes, e.g., cationic liposomes. In one embodiment, the liposomes comprise or more of DC-cholesterol, 1,2-dioleoyl-sn-glycero-3-phosphoethanolamine (DOPE), an ionizable cationic lipid or a lipidoid. In one embodiment, the composition comprises nanoparticles. In one embodiment, the composition comprises a cationic peptide, e.g., poly (l-lysine) (PLL), protamine, or a cell penetrating peptide (CPP). In one embodiment, the composition is targeted to the liver. In one embodiment, the composition comprises collagen type VI receptor, mannose-6-phosphate, galactose or asialoglycoprotein. In one embodiment, the composition is systemically administered. In one embodiment, the composition is orally administered. In one embodiment, the composition is injected. In one embodiment, the seed region comprises 5′AAUGUUU3′ or 5′AATGTTT3′. In one embodiment, the nucleic acid sequence is less than 30 bases in length. In one embodiment, the nucleic acid sequence is less than 25 bases in length. In one embodiment, the nucleic acid sequence is less than 20 bases in length. In one embodiment, the nucleic acid sequence is greater than 10 bases in length. In one embodiment, the composition comprises single stranded RNA comprising the seed region. In one embodiment, the composition comprises RNA comprising a hairpin-loop structure. In one embodiment, the composition comprises double stranded nucleic acid comprising the seed region. In one embodiment, the RNA or one strand of the double stranded nucleic acid comprises an antisense sequence of miRNA-33a-3p. In one embodiment, the RNA or the one strand is less than 70 bases in length. In one embodiment, the RNA or the one strand is less than 50 bases in length. In one embodiment, the RNA or the one strand is less than 25 bases in length. In one embodiment, the RNA or the one strand is greater than 10 bases in length. In one embodiment, the length of the one strand is greater than that of the nucleic acid sequence having the seed region. In one embodiment, the RNA or the one strand is linked to a molecule that enhances cellular uptake, e.g., palmitic acid, a-tocopherol (vitamin E), polyamines such as spermine, lipid docosanyl or stearoyl ligand, anandamide conjugates, or folic acid. In one embodiment, the nucleic acid sequence comprises non-native nucleotides. In one embodiment, the RNA or the one strand comprises non-native nucleotides. In one embodiment, the non-native nucleotide has a modified nucleobase, modified phosphate group or a modified sugar. In one embodiment, the amount of the nucleic acid sequence is about 0.01 mg/kg to about 100 mg/kg. In one embodiment, the amount of the nucleic acid sequence is about 0.05 mg/kg to about 10 mg/kg. In one embodiment, the amount of the nucleic acid sequence is about 10 mg/kg to about 75 mg/kg.

In one embodiment, a method to prevent, inhibit or treat cardiovascular disease in a mammal is provided. In one embodiment, a method to prevent, inhibit or treat high blood pressure in a mammal is provided. In one embodiment, a method to prevent, inhibit or treat diabetes in a mammal is provided. These methods include administering to a mammal in need thereof an effective amount of a composition comprising a nucleic acid sequence comprising a seed region of miRNA-33a-3p. In one embodiment, the mammal is a human. In one embodiment, the disease is coronary heart disease. In one embodiment, the disease is stroke. In one embodiment, the disease is peripheral vascular disease. In one embodiment, the disease is atherosclerosis. In one embodiment, the administration of the composition is in an amount that reduces total cholesterol levels in, e.g., blood, of the mammal. In one embodiment, the administration of the composition is in an amount that amount reduces LDL levels in the mammal. In one embodiment, the composition comprises liposomes, e.g., cationic liposomes. In one embodiment, the liposomes comprise or more of DC-cholesterol, 1,2-dioleoyl-sn-glycero-3-phosphoethanolamine (DOPE), an ionizable cationic lipid or a lipidoid. In one embodiment, the composition comprises nanoparticles. In one embodiment, the composition comprises a cationic peptide, e.g., poly (l-lysine) (PLL), protamine, or a cell penetrating peptide (CPP). In one embodiment, the composition is targeted to the liver. In one embodiment, the composition comprises collagen type VI receptor, mannose-6-phosphate, galactose or asialoglycoprotein. In one embodiment, the composition is systemically administered. In one embodiment, the composition is orally administered. In one embodiment, the composition is injected. In one embodiment, the seed region comprises 5′AAUGUUU3′ or 5′AATGTTT3′. In one embodiment, the nucleic acid sequence is less than 30 bases in length. In one embodiment, the nucleic acid sequence is less than 25 bases in length. In one embodiment, the nucleic acid sequence is less than 20 bases in length. In one embodiment, the nucleic acid sequence is greater than 10 bases in length. In one embodiment, the composition comprises single stranded RNA comprising the seed region. In one embodiment, the composition comprises RNA comprising a hairpin-loop structure. In one embodiment, the composition comprises double stranded nucleic acid comprising the seed region. In one embodiment, the RNA or one strand of the double stranded nucleic acid comprises an antisense sequence of miRNA-33a-3p. In one embodiment, the RNA or the one strand is less than 70 bases in length. In one embodiment, the RNA or the one strand is less than 50 bases in length. In one embodiment, the RNA or the one strand is less than 25 bases in length. In one embodiment, the RNA or the one strand is greater than 10 bases in length. In one embodiment, the length of the one strand is greater than that of the nucleic acid sequence having the seed region. In one embodiment, the RNA or the one strand is linked to a molecule that enhances cellular uptake, e.g., palmitic acid, a-tocopherol (vitamin E), polyamines such as spermine, lipid docosanyl or stearoyl ligand, anandamide conjugates, or folic acid. In one embodiment, the nucleic acid sequence comprises non-native nucleotides. In one embodiment, the RNA or the one strand comprises non-native nucleotides. In one embodiment, the non-native nucleotide has a modified nucleobase, modified phosphate group or a modified sugar. In one embodiment, the amount of the nucleic acid sequence is about 0.01 mg/kg to about 100 mg/kg. In one embodiment, the amount of the nucleic acid sequence is about 0.05 mg/kg to about 10 mg/kg. In one embodiment, the amount of the nucleic acid sequence is about 10 mg/kg to about 75 mg/kg.

The invention will be described by the following non-limiting examples.

### Example 1: Materials and Methods

This Example illustrates some of the materials and methods used in developing the invention.

### Reagents and Plasmids

GW3965 hydrochloride, Simvastatin and Mevalonic Acid Sodium Salt (Sodium Mevalonate) were obtained from Sigma-Aldrich. InSolution™ Cycloheximide, InSolution™ Phorbol-12-myristate-13-acetate (PMA) and Apolipoprotein A-I were obtained from Calbiochem. Dil LDL was purchased from ThermoFisher Scientific and TopFluor Cholesterol from Avanti Polar Lipids. Lipoprotein Deficient Serum, Bovine (LPDS) was procured from Alfa Aesar. The following antibodies were used for Western Blotting and immunofluorescence: LDLR (Abcam, 1:2000 for Western Blotting; 1:250 for immunofluorescence), human PCSK9 (Abcam, 1:2000), mouse PCSK9 (Abcam, 1:1000), ABCA1 (Abcam, 1:1000), B-Actin HRP Conjugate (Cell Signaling, 1:2000), Amersham ECL Rabbit IgG HRP-linked whole Ab from donkey (GE Healthcare Life Sciences, 1:2000), Amersham ECL Mouse IgG HRP-linked whole Ab from sheep (GE Healthcare Life Sciences, 1:2000) and Goat Anti-Rabbit Alexa Fluor 488 (Abcam, 1:1000). The human PCSK9 ORF clone with a C-terminal Flag tag in a pReceiver-M13 Expression Clone, lacking 5′- or 3′-UTRs, was obtained from GeneCopoeia. The following vectors were used for luciferase assays: (1) pLightSwitch_3UTR vector from Switchgear Genomics with human Mylip (IDOL) or human PCSK9 3′-UTRs cloned downstream of the RenSP luciferase gene, was used along with pSV—B-Galactosidase Control Vector from Promega to validate the binding of hsa-miR-33a-3p on human Mylip (IDOL) and PCSK9 3′-UTRs. (2) The pEZX-MT05 dual reporter vector from GeneCopoeia that encodes two secreted reporter enzymes, namely Gaussia Luciferase (GLuc) and Secreted Alkaline Phosphatase (SEAP) was used for validating the association of miR-33a-5p with human Mylip, mouse Mylip and mouse PCSK9 3′-UTRs. (3) The pEZXMT06 dual reporter vector encoding Firefly Luciferase and Renilla Luciferase was used for testing the binding of miR-33-3p on mouse Mylip. microRNA binding sites on target sequences were mutated by means of site-directed mutagenesis of two bases in each case using the QuikChange Lightning Site-Directed Mutagenesis Kit (Agilent). All mutations introduced were confirmed by Sanger DNA sequencing. Primers used for Site-Directed Mutagenesis and sequencing were procured from Integrated DNA Technologies, Illinois, USA.

When using a promoter-driven expression vector to express miR-33a-3p, any constructs containing the mature of miR-33a-3p as part of a shRNA-like stem loop expressing the primary or precursor form of miR-33a-3p (seed sequence: CAAUGUUU; DNA form: CAATGTTT) were effective as an alternative strategy to miR-33a-3p mimics.

### Cell Culture and Transfection

HepG2, HEK 293T, THP-1 and J774 cell lines were procured from American Type Culture Collection (ATCC). Plateable Cryopreserved Human Primary Hepatocytes were purchased from Gibco. HepG2, HEK 293T and J774 cells were maintained in Dulbecco's Modified Eagle's medium (DMEM) with 10% FBS and 1% Penicillin-Streptomycin-Glutamine in 10 cm dishes in a humidified incubator at 37° C. and 5% CO₂. THP-1 cells were grown in Roswell Park Memorial Institute (RPMI) 1640 media containing L-Glutamine, supplemented with 10% FBS, 1% Penicillin-Streptomycin-Glutamine, 0.05 mM β-mercaptoethanol and 1 mM Sodium Pyruvate in a humidified incubator at 37° C. and 5% CO₂. Differentiation of THP-1 cells to macrophages was induced by maintaining them in media containing 100 nM PMA for 72 hours. Cryopreserved Primary Hepatocytes were thawed in a 37° C. water bath for <2 minutes, transferred to CHRM media, spun down and resuspended in plating media (William's Medium E without phenol red, supplemented with serum-containing Hepatocyte Plating Supplement Pack (Gibco)). After determining cell viability, cells were seeded at a density of 1×10⁶ cells/ml in collagen-coated 6-well plates. To apply the low sterol condition, cells were transferred to basal growth media supplemented with 10% LPDS in place of FBS, 5 μM Simvastatin and 100 μM Sodium Mevalonate. To induce the LXR condition, cells were transferred to basal growth media with 10% FBS and 1 μM GW3965 hydrochloride. For microRNA overexpression and knockdown, cells were transiently transfected with Ambion Pre-miR miRNA Precursors (ThermoFisher Scientific) and miRCURY LNA™ Power microRNA inhibitor (Exiqon), respectively. The corresponding scrambled controls were also transfected to serve as negative controls. HepG2 and HEK 293T cells were transfected using Lipofectamine 3000 (ThermoFisher Scientific), while J774 and THP-1 macrophages were transfected using HiPerFect Transfection Reagent (Qiagen). Targefect-Hepatocyte Reagent (Targeting Systems) was used to transfect Human Primary Hepatocytes.

### RNA Isolation and Quantitative Real-Time PCR

microRNA and mRNA PCR were performed using total RNA extracted from cultured cells and animal tissues by means of the mirVana™ miRNA Isolation Kit, with phenol (ThermoFisher Scientific). TissueLyser II sample disruptor (Qiagen) was used to disrupt and homogenize animal tissues. Concentration-adjusted total RNA was reverse transcribed into cDNA using the Universal cDNA Synthesis Kit II (Exiqon) for microRNAs, or iScript Reverse Transcription Supermix (Bio-Rad) for mRNAs. qRT PCR was performed on an Applied Biosystems 7500 Real-Time PCR System (ThermoFisher Scientific) using ExiLENT SYBR® Green master mix (Exiqon) for microRNAs, or PowerUp SYBR Green Master Mix (ThermoFisher Scientific) for mRNAs. Normalization was done using the following genes as reference: miR-423-3p for human and mouse microRNAs, HMBS or B2M for human mRNAs, and HPRT or B2M for mouse mRNAs. The primers used were obtained from Exiqon (for microRNAs) and Integrated DNA Technologies (for mRNAs). Primer sequences are available upon request.

### Western Blotting

Protein extracts from cells and tissues were prepared using Minute™ Total Protein Extraction Kit (Invent Biotechnologies) following the native extraction procedure. 1× of Halt™ Protease and Phosphatase Inhibitor Cocktail (ThermoFisher Scientific) was added to the lysis buffer prior to lysis. Animal tissues were homogenized using a TissueLyser II sample disruptor (Qiagen) in native lysis buffer before protein extraction. Concentrations were measured with the DC™ Protein Assay (Bio-Rad). Bovine Serum Albumin (BSA) standards of known concentrations were used to plot a standard curve and protein concentration in each sample was determined by comparing its absorbance value to the standard curve by means of polynomial regression analysis.

For all samples, 30 μg of total protein was mixed with Laemmli Buffer containing β-mercaptoethanol and separated by SDS-PAGE using AnykD Mini-PROTEAN TGX Precast Protein Gels (Bio-Rad). Proteins were transferred onto Immun-Blot® PVDF Membrane, 0.2 μM (Bio-Rad) by Wet Transfer for 1 hour at room temperature, blocked with 5% milk in PBS for 1 hour and probed with the indicated antibodies. Bands were visualized using Pierce ECL 2 Western Blotting Substrate (Fisher Scientific) on a Geliance 600 Chemi Imaging System (PerkinElmer), and densitometry was carried out using the Image Studio™ Lite software, version 5 (Li-Cor).

### Cycloheximide Degradation Assay

HepG2 cells were seeded in 6-well plates, transferred to media containing GW3965 hydrochloride and transfected with miR-33a-3p or negative control precursors. After 48 hours of incubation, media was replaced with fresh one containing 100 μg/ml Cycloheximide. Subsequently, cells were harvested and protein lysates prepared at 0, 4 and 16 hour time points. Lysates were assayed for ABCA1 protein levels by Western Blotting.

### Secreted PCSK9 ELISA

PCSK9 secreted into the cell culture media was measured by means of a solid phase sandwich ELISA using the Human PCSK9 Quantikine ELISA Kit (R&D Systems), following the manufacturer's instructions. Cell culture supernatants collected from samples, standards and controls were all assayed in duplicate and the mean was calculated. Mean of the blank was subtracted from those of the standards and samples. PCSK9 concentration in each sample was determined by comparing its absorbance value to a standard curve through polynomial regression analysis.

### LDL Uptake Assay

HepG2 cells were first seeded in 6-well plates and subsequently transferred to low sterol media the next day before being transfected with microRNA precursors or inhibitors. After the incubation period, cells were washed two times with PBS and fresh low sterol media containing 5 μg/ml Dil LDL (ThermoFisher Scientific) was added. Media was removed after 60 minutes and cells were washed four times with PBS before preparation of cell lysates. Equal volumes of the cleared lysates were loaded in triplicate on a 96-well clear bottom black assay plate (Corning). Fluorescence intensity was measured at an Excitation/Emission of 554/571 nm, with a cut-off of 550 nm, using FlexStation 3 Multi-Mode Microplate Reader (Molecular Devices). Blank-subtracted fluorescence values were normalized to total protein content measured separately.

### Cholesterol Efflux Assay

J774 cells were seeded in a 24-well plate at high density in DMEM+10% FBS media without antibiotics. Next day, cells were transfected with microRNA precursors, and after 24 hours, 12.5 μM TopFluor Cholesterol was added to the cells. After a further incubation period of 24 hours, media was removed and cells were washed four times with PBS. Fresh DMEM+10% FBS media containing 20 μg/ml Apo AI was added and cells were incubated for 5 hours. Both the media and cells were collected post incubation. Media was spun down to remove floating cells, while the attached cell monolayer was washed four times with PBS and lysed to prepare protein extracts. To measure fluorescence content in the media and cells, samples were loaded in triplicate on a 96-well clear bottom black assay plate (Corning). Fluorescence intensity was measured at an Excitation/Emission of 490/520 nm, with a cut-off of 495 nm, using FlexStation 3 Multi-Mode Microplate Reader (Molecular Devices). Cellular protein concentration was measured separately and cellular fluorescence values were normalized to total protein content. Percentage cholesterol efflux was calculated by dividing the media fluorescence by the sum of media and cellular fluorescence.

### Luciferase Assays

HEK 293T cells were seeded in 24-well collagen-coated plates and cotransfected with the reporter construct(s) along with 50 nM of microRNA precursors or scrambled control precursors. For assays involving Renilla luciferase and B-Galactosidase as the primary and secondary reporters, respectively, the β-Galactosidase Enzyme Assay System with Reporter Lysis Buffer kit (Promega) was used to lyse cells and measure β-Galactosidase expression. The same lysate was also used to quantify Renilla Luciferase activity by means of the Renilla-Glo® Luciferase Assay System (Promega). Then, Renilla luminescence units were normalized to the β-Galactosidase assay absorbance values. For assays involving the Gaussia Luciferase and Secreted Alkaline Phosphatase (GLuc-SEAP) dual reporter construct, cell culture media was collected 48 hours post transfection and GLuc and SEAP activities present in the media were sequentially measured using the Secrete-Pair™ Dual Luminescence Assay kit (GeneCopoeia). Measured values of the GLuc reporter gene were normalized to SEAP luminescence intensities. In assays involving Firefly and Renilla luciferase dual reporter constructs, cells were lysed 48 hours after transfection and both luciferase activities quantified using the Dual-Luciferase® Reporter Assay System (Promega). Firefly luminescence was normalized to Renilla luminescence values.

### Immunofluorescence

HepG2 cells were grown in 24-well Glass Bottom Plates with No. 1.5 Coverslip (MatTek Corporation) and transfected with 50 nM microRNA precursors under the low sterol condition. After 48 hours, cells were washed two times with PBS, fresh low sterol media containing 5 μg/ml Dil LDL (ThermoFisher Scientific) was added and cells were incubated again for 60 minutes. Subsequently, media was removed, cells were washed four times with PBS and then fixed in 1 ml of 4% Paraformaldehyde in PBS for 20 minutes at room temperature. Cells were then washed three times for 10 minutes each with PBS. Blocking was performed with 1 ml of Normal Goat Serum (10%) in PBS (ThermoFisher Scientific) for 30 minutes. Anti-LDLR primary antibody was added at a dilution of 1:250 in Normal Goat Serum and incubated overnight at 4° C. Next day, cells were washed three times for 30 minutes each with PBS and Goat Anti-Rabbit Alexa Fluor 488 secondary antibody was added at a dilution of 1:1000 in Normal Goat Serum. After an incubation period of 1 h at room temperature, cells were washed again two times for 30 minutes each with PBS. A Microscope Slide Cover Glass: Circular 12 mm diameter, #1 thickness (Propper Manufacturing Co., Inc.) was then mounted onto the cells with VECTASHIELD HardSet Antifade Mounting Medium with DAPI (Vector Laboratories). Plates were left in the dark at 4° C. for the mounting medium to harden. A no-primary antibody negative control was included in the assay. Imaging was done using a Zeiss LSM 880 Confocal Microscope with an oil-immersion 63× Objective under 2× Zoom. EGFP, mCherry and DAPI channels were imaged keeping laser intensity, gain and other parameters constant across channels. Zen 2.3 SP1 software was used to collect and process raw images.

### Statistical Analysis

All experiments were performed in triplicate and the mean of the three replicate values+standard deviation (S.D.) are presented. Error bars represent standard deviation. Statistical comparison between two groups was conducted by means of a two-tailed type I Student's t test. P-values≤0.05 were considered significant and are summarized with one asterisk (*), P-values≤0.01 are summarized with two asterisks (**) and P-values≤0.001 are summarized with three asterisks (***).

### Mouse Injections, Sample Collection and Analysis

All mice procedures were approved by the Weill Cornell Medicine Institutional Animal Care and Use Committee. 28-week-old male C57BL/6J DIO mice (Stock #: 380050 Black 6 DIO) kept on a high-fat diet with 60% kcal from milk fat (HFD; D12492, Research Diets) were purchased from the Jackson Laboratory, Bar Harbor, ME, USA. The mice were maintained on HFD until they were 36-weeks-old and reached a body weight ≥50 g. Then the animals were switched to a Western-type diet with 45% kcal from milk fat (D12451, Research Diets) for two weeks. After that the Western-type diet was supplemented with 1% cholesterol (D09071604, Research Diets) for two more weeks prior to and during the treatment. Mice were grouped into two groups of five mice each with the average weight of both groups being the same. miR-33-3p HPLC/In Vivo Ready mirVana® miRNA mimic and mirVana™ miRNA Mimic Negative Control #1 were complexed with Invivofectamine 3.0 reagent following the manufacturer's instructions (ThermoFisher Scientific). Individual mice belonging to each group received either the miR-33-3p mimic or control mimic at 1 mg/kg dose in 200 μl injection volume per 50 g mouse via tail vein injections. Mice were treated on days zero and four at the same time of the day and sacrificed by CO₂ euthanasia on day eight following five hours of fasting.

After sacrificing, 1 ml of blood was obtained from each mouse by right ventricular puncture in a serum separator tube (BD, Ref.365967). Blood was centrifuged at 6000 RPM for 1.5 minutes to obtain serum, which was frozen at −80° C. until analysis. Serum ALT, AST, glucose, total cholesterol, triglycerides, HDL and LDL were measured at The Laboratory of Comparative Pathology and Mouse Phenotyping, New York, USA. FPLC analysis of pooled serum was carried out as described (Najafi-Shoushtari et al., 2010). Peritoneal Macrophages were collected by injecting 15 ml of ice cold PBS into the peritoneal cavity and then moving the mice back and forth. Around 12-16 ml of the PBS containing macrophages was aspirated out using a 20 ml syringe, followed by retrieval of the macrophages by centrifugation for downstream RNA and protein analyses. Liver pieces were excised out and either snap frozen in liquid nitrogen for protein analysis or immersed in RNAlater Solution (ThermoFisher Scientific) for RNA isolation before being transferred to −80° C. For immunohistochemistry, a piece of the liver was embedded in Tissue-Tek OCT compound (Sakura Finetek, Ref. 4583), cryopreserved in a cryomold (Sakura Finetek, Ref. 4566) and stored at −80° C. until sectioning.

### Immunohistochemistry

Liver tissues affixed in the cryomold were sectioned to 7 μm using a Cryostat (LEICA, CM-3050-S) and the sections were immobilized onto Superfrost™plus microscope slides (ThermoFisher Scientific, Cat. #4951PLUS4). The sections were stained with Oil Red O (ORO) dye according to Mehlem, A. et al (2013). Briefly, 2.5 g of ORO (Sigma, Cat. #00625) was added to 400 ml of 99% isopropyl alcohol (Sigma, Cat. #278475) and mixed on a magnetic stirrer for two hours at room temperature to prepare a stock solution. Then, about 1.5 parts of the ORO stock solution was mixed with one part of distilled water and allowed to stand for 10 minutes at 4° C. This working solution was filtered using a 0.2 μm syringe filter (VWR International, Cat. #: 28145-477) to remove any precipitates. Liver sections were then incubated with the ORO working solution for 10 minutes at room temperature and counterstained with hematoxylin (EMS, Cat. #: 26503-04) for 15 seconds. Afterwards, the sections were rinsed under running tap water for 10 minutes and mounted with a water-soluble mounting media and cover slip. The stained sections were photographed using Zeiss Axio Scope A1 within 3-4 hours to avoid precipitation of the ORO dye. Images were analyzed for adipocyte cell size using Image J along with adipocyte tools as macros (see website at dev.mri.cnrs.fr/projects/imagej-macros/wiki/Adipocytes_Tool)

### BMDM Culture and Ago2 PAR-CLIP Analysis

Animals were sacrificed using carbon dioxide and bone marrow cells from mouse femur and tibia were collected by flushing through PBS with a 23-gauge needle. Bone marrow cells were cultured in one 150 mm Petri dishes with complete DMEM and 20% of L929 cell culture medium for 6 days (Zhang et al., 2012). BMDM for PAR-CLIP experiment were cultured with 100 μM 4-thiouridine (Sigma) for 16 hours, washed with PBS and UV-crosslinked. Two sets of BMDMs (7 mouse for each set) were placed on ice and radiated uncovered with 0.15 J/cm2 total energy of 365 nm UV light in a Stratalinker (Invitrogen). BMDM were then harvested by incubation at 37° C. for 10 minutes with 0.2 mM EDTA in PBS, washed twice with PBS and frozen at −80° C. The details of the PAR-CLIP protocol were described previously (Sorrentino et al., 2013; Seidah et al., 2014) with several minor modifications as follows. A mouse monoclonal anti-mouse AGO2 antibody (WAKO) was used for immunoprecipitation of miRNAs (Ago2 IP). The expected radioactive labeled RNA-protein complex, which is around 100 kD, was observed by autoradiography. The cross-linked miRNA or mRNA fragments were isolated for cDNA library preparation and deep sequencing. The following were used from an Illumina TruSeq Small RNA Sample preparation Kit: 3′ adaptors, 5′ adaptors, RT primers, PCR primers. PAR-CLIP small RNA libraries from 2 samples were sequenced for 45 cycles on Illumina HiSeq 2000 platform (Illumina).

For Ago2 PAR-CLIP library, PARalyzer was used to identify binding sites as described previously (Goldstein & Brown, 2009). Briefly, reads that aligned to a mouse MM9 unique genomic location, after correction of T to C mismatches and overlapped by at least one nucleotide were grouped together. Read groups were analyzed for T to C conversions and nucleotide strings containing a greater likelihood of converted T to Cs than non-converted Ts were extracted as clusters. AGO2 PAR-CLIP clusters are defined as having at least 25 reads, exclude genomic repeat regions, and meeting the T to C conversion criteria. Only groups of overlapping sequence reads that exceed a threshold of ≥50% T to C conversion frequency (except miRNAs) were considered. To compare 2 Ago2 PAR-CLIP datasets, we merged the PAR-CLIP clusters as follows. The overlapping cluster should be least 18 nucleotide in size and the overlapping sequence between samples should have more than 15 nucleotide. If there are multiple sites in adjacent areas in one sample and the sites cannot be distinguished from one another, the reads of each cluster will be combined and represented as the total read number for this integrated large cluster sequence. The sites were selected from more than 100 reads in combined samples (>98% overlapping) for further miRNA prediction. Clusters that overlapped with predicted miRNA binding site from TargetScan database (TargetScan 6.2, mouse non-conserved and conserved predictions) were identified using custom scripts. Since TargetScan uses canonical seed match sites (≥7mer1A, i.e. nucleotide 2-7 match with an A across from position one of the mature miRNA), the PITA algorithm was used to search for miRNA target sites allowing either 1 G: U wobble or 1 mismatch in the 7-8 nucleotide seed site (Segal Lab of Computational Biology) when no TargetScan predictions were found for a given cluster. Overall PAR-CLIP data, miRNA sequencing data and mRNA sequencing data were published previously (Scotti et al., 2013) and sequencing data have been submitted to the GEO database with the following accession numbers: GSE63199.

### Example 2: miR-33a-3p and miR-33a-5p, Along with their SREBP-2, Promote LDLR Expression

HepG2 cells were first seeded in 6-well plates and subsequently transferred to different media the next day. The media conditions used included LPDS (Lipoprotein Deficient Serum) media, FBS (normal conditions) media, and media with an LXR-agonist (GW3965). Expression levels of miR-33a-3p, miR-33a-5p, ABCA1, IDOL, SREBP2, LDLR, PCSK9, and HMGCR in the cells were evaluated.

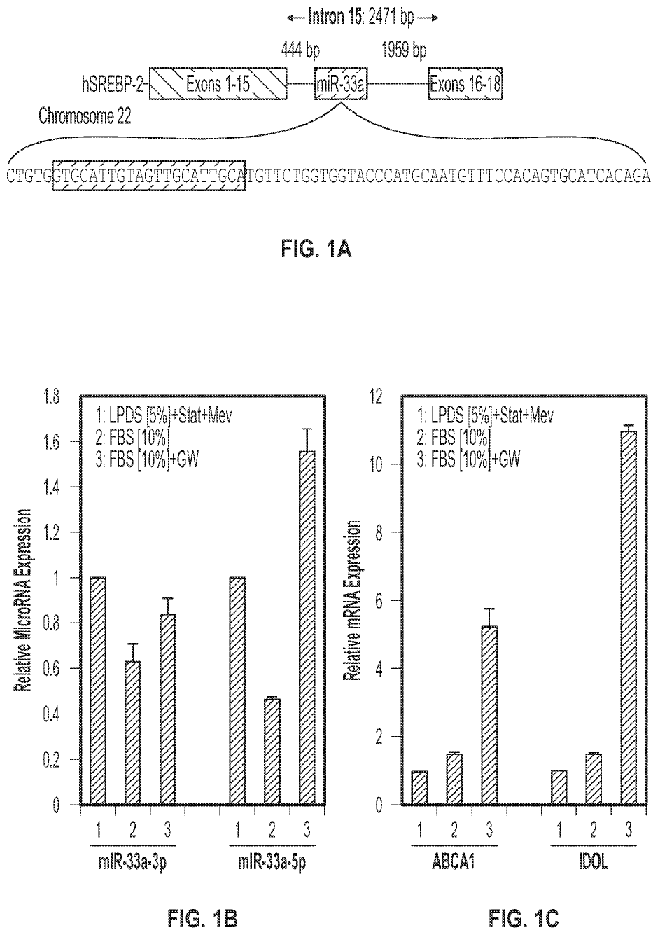

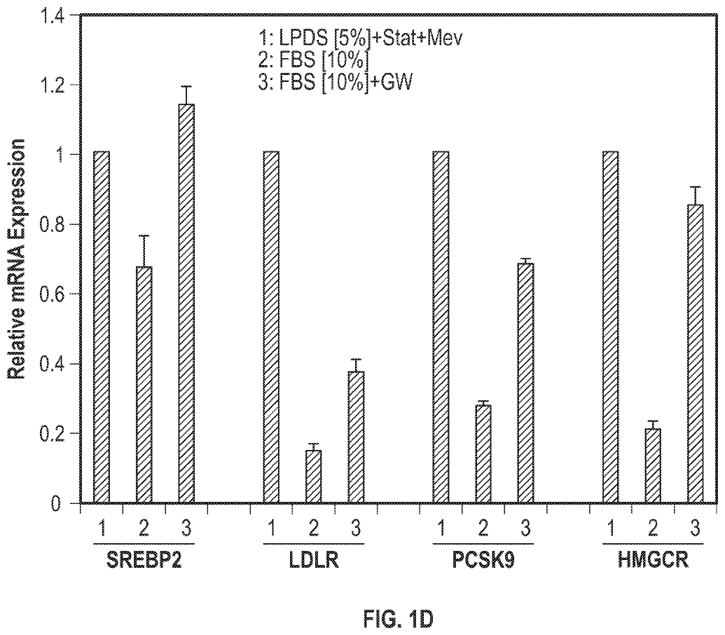

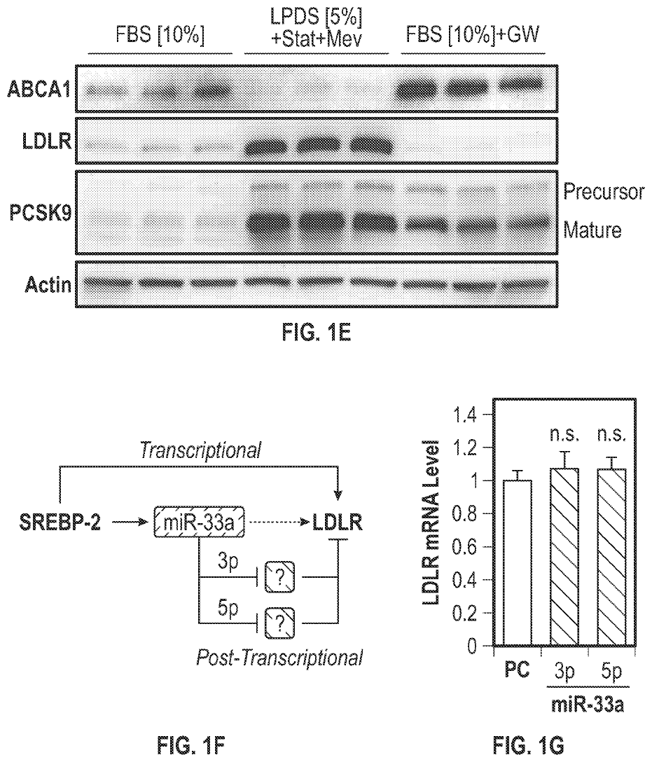

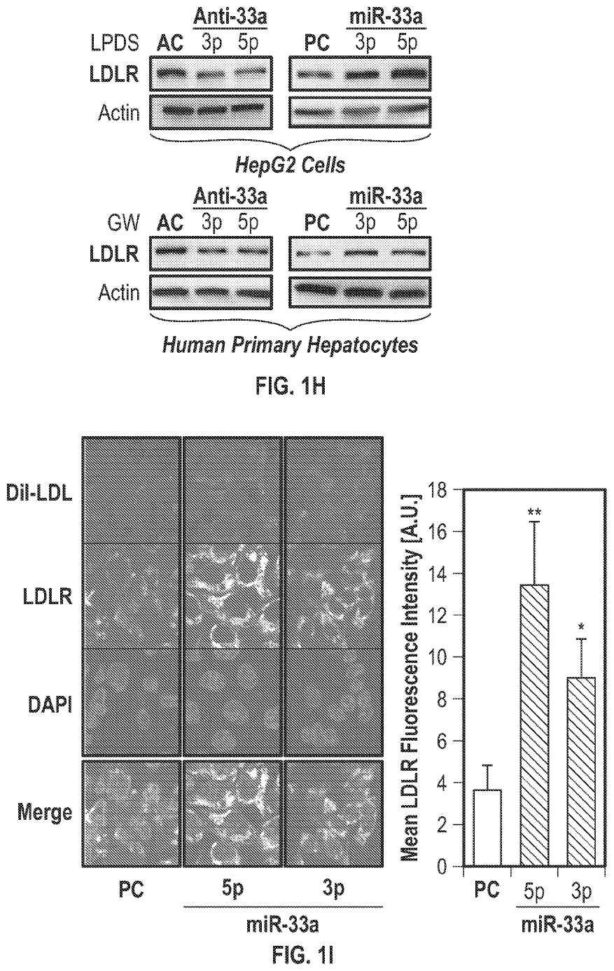

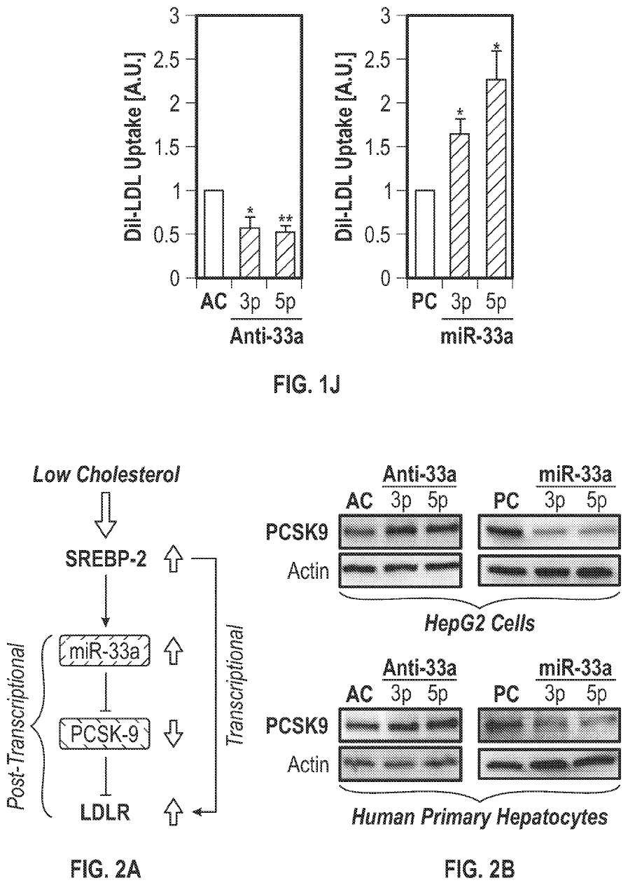

The microRNA duplex comprising miR-33a-3p and miR-33a-5p strands exhibit a similar trends of activation or repression in response to modulated cellular sterol levels (FIG. 1B-1E). While both strands remain well-detectable in liver cells, miR-33a-5p may serve as the guide strand given its relatively higher abundance. As illustrated in FIG. 1B, miR-33a-3p and miR-33a-5p expression levels increased under low sterol conditions. Such low sterol conditions included use of lipid depleted (LPDS) media that contained simvastatin and sodium mevalonate. Expression levels of liver cells in normal conditions (FBS) and media containing GW3965 (GW) were also evaluated. GW 3965 hydrochloride is a non-steroidal agonist for liver X receptor (LXR). FIG. 1C illustrates expression of ABCA1 and IDOL (“Inducible Degrader of the LDL receptor”) under the low sterol conditions (LDPS+simvastatin+sodium mevalonate) conditions, normal conditions (FBS), and in media containing GW3965 (GW). As illustrated, ABACA1 and IDOL expression levels are lower under low sterol conditions than under GW conditions activate LXR. FIG. 1D shows that SREBP-2, LDLR, PCSK9, and HMGCR expression levels are increased under the low sterol conditions. FIG. 1E illustrates that ABCA1 protein levels are lower but LDLR and PCSK9 protein levels are increased under the low sterol conditions. FIG. 1F schematically summarizes the transcriptional relationships between SREP-2, LDLR, and miR-33a, and reflect the current uncertainties regarding miR-33a effects on LDLR.

As illustrated in FIG. 1G, addition of miR-33a-3p and miR-33a-5p had no significant effects on LDLR mRNA levels in cultured hepatic cells, but the right panels of FIG. 1H show that addition of miR-33a-3p and miR-33a-5p increased LDLR protein levels relative to a precursor control (PC).

To examine whether miR-33a might be functionally linked to the LDLR pathway, antisense oligonucleotide-mediated knockdown of miR-33a-3p and/or miR-33a-5p was performed in liver cells, including HepG2 hepatoma and isolated primary human hepatocytes. As shown in the left panels of FIG. 1H, miR-33a-3p or miR-33a-5p antisense depletion under induced SREBP-2 (LPDS) or LXR (GW) conditions reduced LDLR protein expression. Again, the effects on LDLR protein expression were not accompanied by concomitant changes in LDLR mRNA (FIG. 1G, some data not shown).

Conversely, internalization of fluorescently labeled LDL (Dil-LDL) into HepG2 cells was strongly increased upon transfection of miR-33a-3p and miR-33a-5p as analyzed by immunofluorescence and cellular Dil-LDL measurement (FIG. 1I-1J). Antisense antagonism of either miR-33a strand, however, caused a significant decrease in Dil-LDL uptake (FIG. 1J), as compared to scrambled control (AC) anti-miR-treated cells.

These result indicate that miR-33a mutually stimulates LDLR expression and activity downstream of SREBP-2-induced transcription.

### Example 3: miR-33a-3p and miR-33a-5p Directly Inhibit PCSK9 Expression and Function

This Example describes experiments designed to determine how miR-33a mutually stimulates LDLR expression and activity downstream of SREBP-2-induced transcription. The role of two regulators of LDLR, PCSK9 and IDOL, were investigated as potential direct targets of miR-33a given their reciprocal function during LDL uptake (Sorrentino et al., 2013; Seidah et al., 2014).

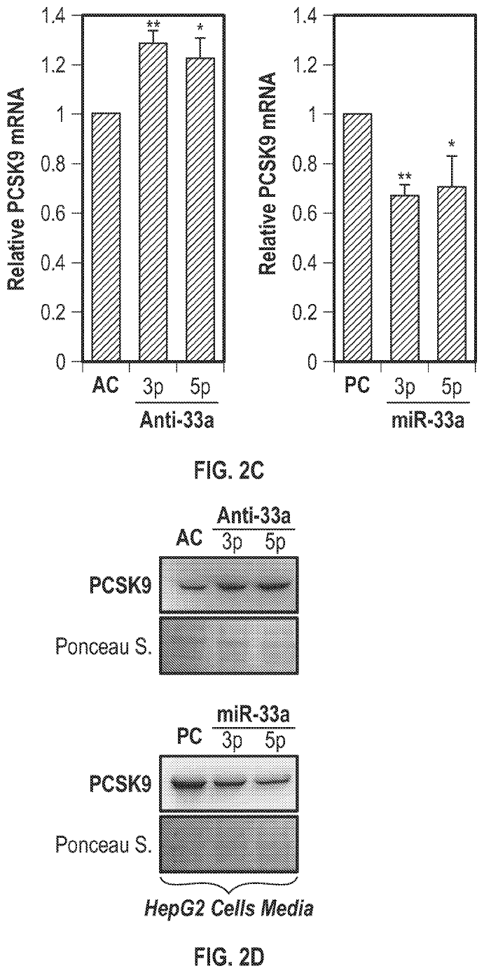

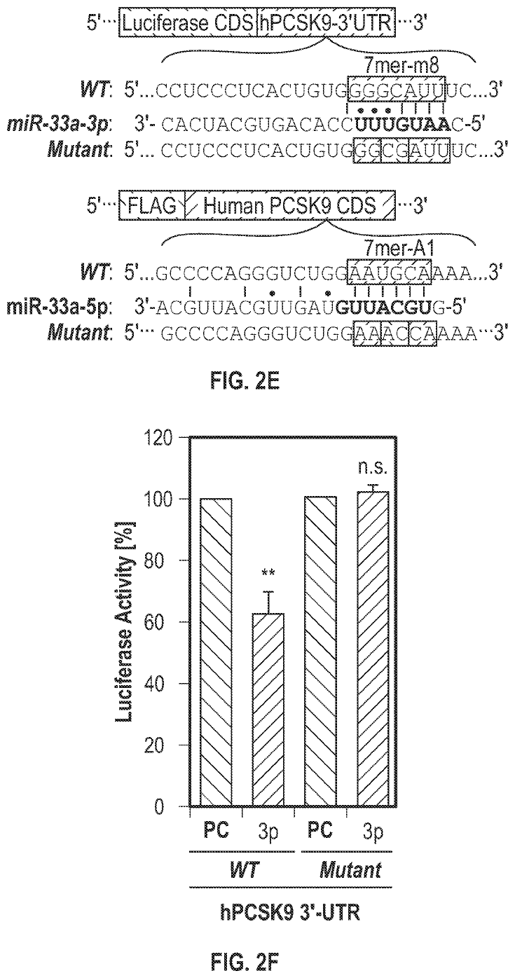

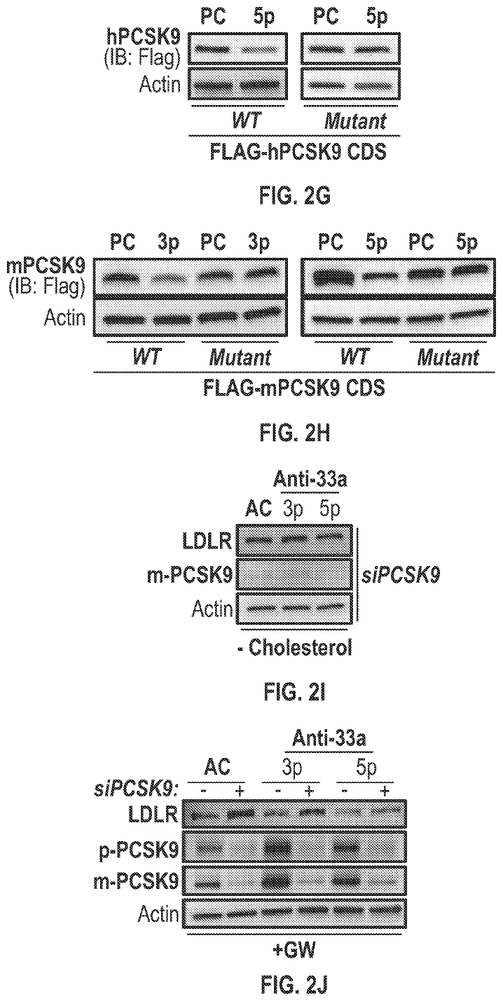

First, miR-33a directed inhibition of PCSK9 was addressed. As shown in FIG. 2A-2D, human hepatocytes treated with antisense miR-33a-3p/5p under lipid-depleted or LXR-induced conditions exhibited increased expression of PCSK9 at both the mRNA and protein levels.

The levels of the mature form of PCSK9 that binds to extracellular domain of LDLR upon secretion inversely correlates with plasma LDL-C levels and reflects PCSK9 activity. In examining miR-33a-3p/5p effects on the secretion of PCSK9, slight but significant increased secretion of PCSK9 was observed from HepG2 cells into the cell media with antisense miR-33a-3p/5p (FIG. 2D). Reciprocally, exogenous overexpression of miR-33a-3p or miR-33a-5p led to reduced PCSK9 secretion (FIG. 2C-2D).

To elucidate whether miR-33a-directed inhibition of PCSK9 led to the elevated LDLR levels, siRNA-mediated knockdown of PCSK9 was integrated with the effects of anti-miR-33a-3p/5p treatment on LDLR in HepG2 cells. PCSK9 knockdown blocked the enhanced LDLR expression levels in miR-33a-3p/5p-depleted cells, indicating that LDLR was upregulated by miR-33 in a PCSK9-dependent manner (FIG. 2I-2J).

Direct microRNA target repression occurs upon microRNA recognition and binding at the 3′ untranslated region (UTR), as well as to a lesser extent at the coding domain sequence (CDS) (Brümmer & Hausser, 2014). To examine if miR-33a directly binds the human PCSK9 transcript for inhibition, miR-33a-5p and miR-33a-3p were screened for a potential 7mer-m8 binding site for miR-33a-3p, including a mutant miR-33a-3p with a G-U wobble base. Such a 7mer-m8 binding site for miR-33a-3p was identified in the PCSK9 3′-UTR (FIG. 2E). A potential 7mer-A1 binding site for miR-33a-5p was also found within the PCSK9 CDS (FIG. 2E). In the mouse Pcsk9, putative miR-33a bindings sites were identified only within the CDS, two of which were miR-33a-5p binding sites (7mer-m8 and 7mer-A1), and one was a miR-33-3p offset-6mer site.

A luciferase reporter containing the human PCSK9 3′UTR with the miR-33a-3p binding site displayed miR-33a-3p-dependent repression of luciferase expression (FIG. 2F). However, the mutant miR-33-3p (with two nucleotide substitutions in its seed region) did not reduce luciferase expression (FIG. 2F). Hence, mutation of the seed region of miR-33-3p abolished its regulation of expression from the PCSK9 3′UTR.

Ectopic expression of FLAG-tagged human or mouse PCSK9 cDNA lacking the 3′-UTR, exhibited miR-33a-3p and miR-33a-5p-dependent repression, but mutating individual binding sites abolished the decreased expression of FLAG-tagged PCSK9 (FIG. 2H, human; FIG. 2I, mouse).

### Example 4: miR-33a-3p Directly Inhibits the IDOL-Pathway

This Example describes experiments designed to evaluate a role for miR-33a in LXR-induced IDOL activation, which can be a major cellular mechanism for LDLR regulation.

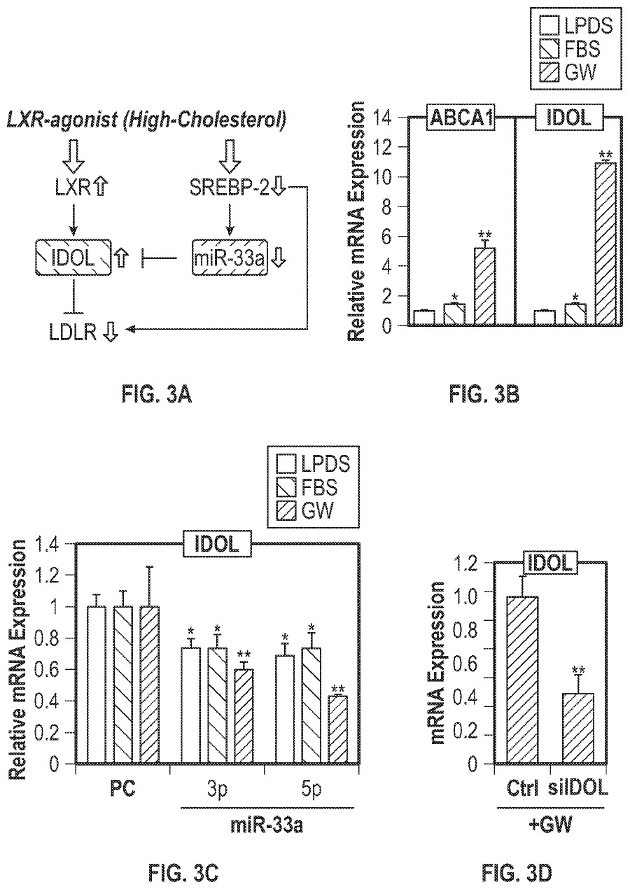

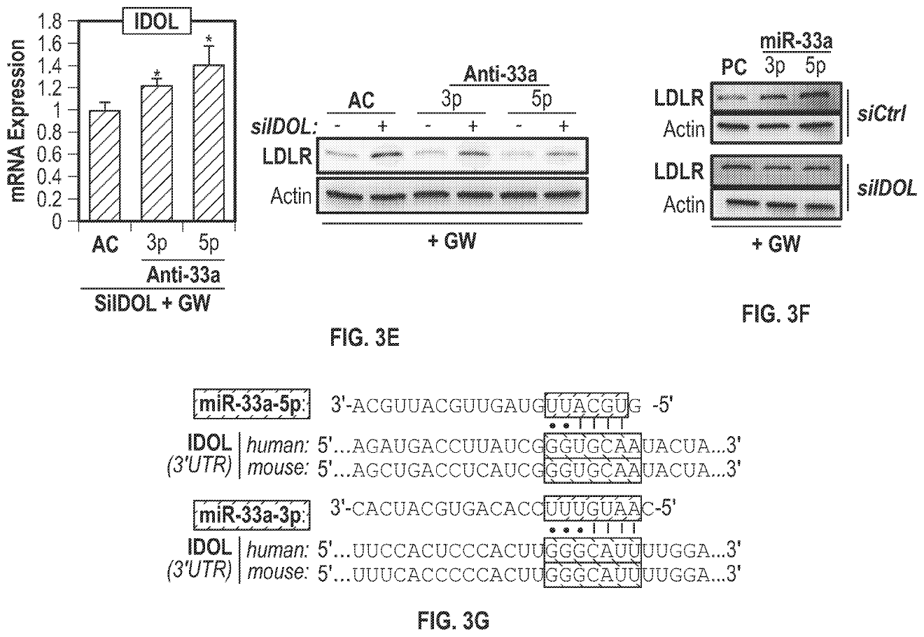

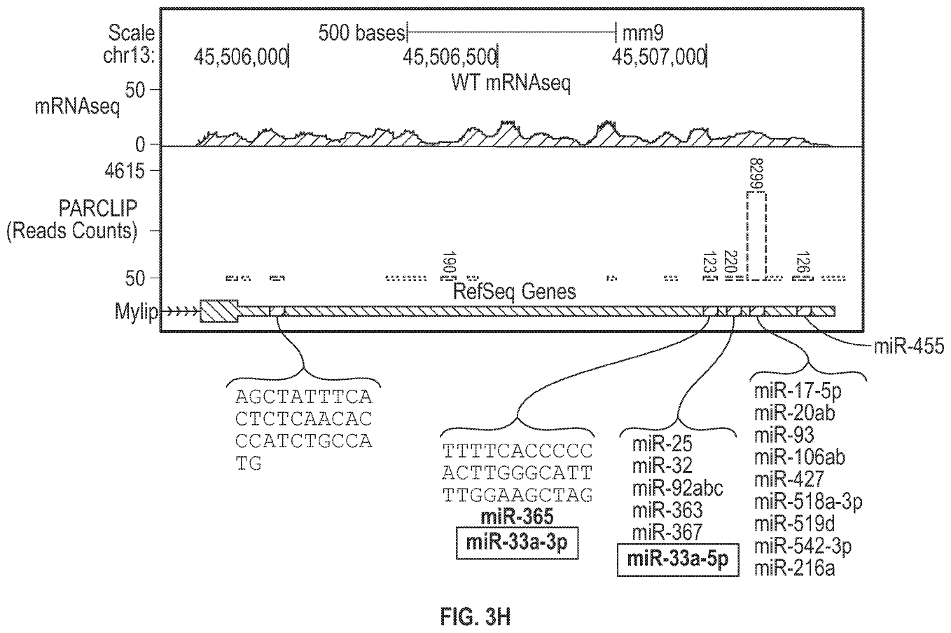

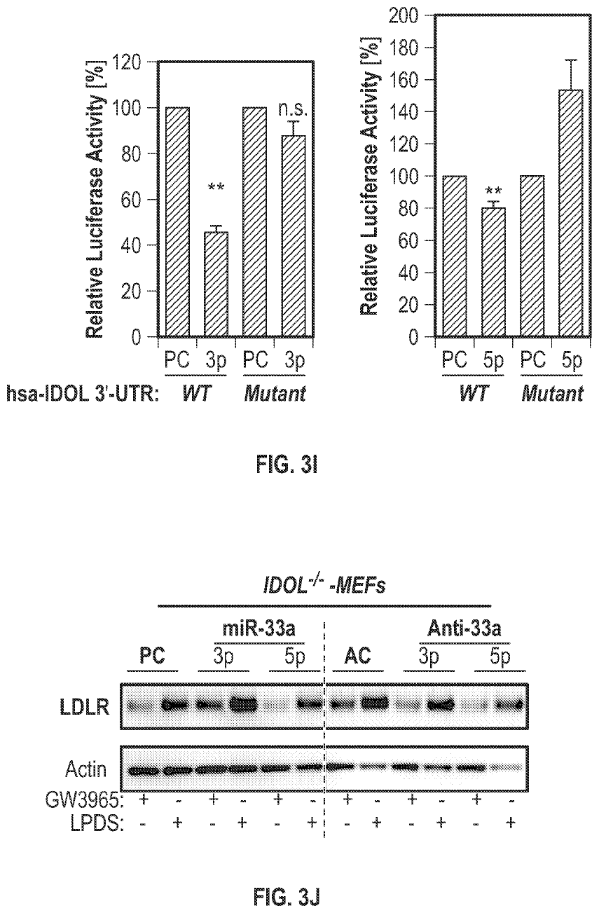

Consistent with activation of IDOL by LXR (FIG. 3A-3B), miR-33a-3p and miR-33a-5p overexpression significantly lowered IDOL expression in HepG2 cells under LXR-agonist-treated conditions, where GW3965 was present (FIG. 3C). Of note, the impact of miR-33a on IDOL protein failed in view of the labile nature of the IDOL protein, which has been reported when using commercially available anti-IDOL antibodies (Scotti et al., 2012; Scotti et al., 2011) (data not shown).

Antisense repression of miR-33a-3p and miR-33a-5p, on the other hand, failed to increase IDOL mRNA. However, following GW3965-induced expression of IDOL, concomitant reduction of IDOL gene expression using anti-IDOL siRNA (FIG. 3D) with antisense miR-33a-3p/5p oligos in HepG2 cells, led to de-repression of LDLR (FIG. 3E).

IDOL antisense knockdown following GW3965-induced expression of IDOL resulted in a significant increases in LDLR levels and reversed miR-33a-3p or miR-33a-5p dependent augmentation of LDLR (FIG. 3F). In contrast, anti-miR-mediated repression of LDLR expression was partially rendered ineffective in the presence of siRNA-mediated knockdown of IDOL (FIG. 3F).

Experiments were then performed to determine whether IDOL is a direct miR-33a target. IDOL harbors two potential miR-33a bindings sites within its 3′UTR, with miR-33a seed matches conserved in humans and mice (FIG. 3G). Consistent with this hypothesis, unbiased AGO2 PAR-CLIP analysis of mouse bone marrow-derived macrophages (BMDMs) revealed AGO2-bound RNA fragments from the 3′UTR of Idol mRNA sequence that matched both miR-33a-3p and miR-33a-5p binding sites (FIG. 3H). Indeed, luciferase reporters bearing both predicted binding sites of either human or mouse IDOL-3′UTR, downstream of the luciferase gene, exhibited reduced miR-33a-3p and 5p-dependent expression (FIG. 3I). Mutation of individual sites abolished or partially rescued this regulation (FIG. 3I).

Hepatic IDOL minimally affects LDLR expression in mice, unless its expression is ectopically elevated by more than a hundred-fold (Hong et al., 2014). To assess whether miR-33a regulation of IDOL follows the same regulatory path in murine cells, the expression of LDLR protein in response to miR-33a modulation in wild-type and IDOL-deficient mouse embryonic fibroblasts (MEFs) was quantitated. Idol′-MEFs exhibit a relatively higher LDLR expression profile (Fairall et al., 2011) that can be further induced independent of IDOL, under a lipid-depleted conditions, but not acutely suppressed with the synthetic LXR-agonist GW3965. The miR-33a modulatory effects on LDLR under GW conditions were found to be present regardless of the presence or absence of Idol, indicating these miRNAs largely regulate LDLR independently of IDOL in mice (FIG. 3J). LXR activation ultimately leads to upregulation of SREBP-2 pathway. Consistent with such upregulation, the data provided herein shows that miR-33a functions to protect LDLR from degradation by direct repression of PCSK9 and IDOL.

### Example 5: miR-33a-3p Increases ABCA1 Protein Levels

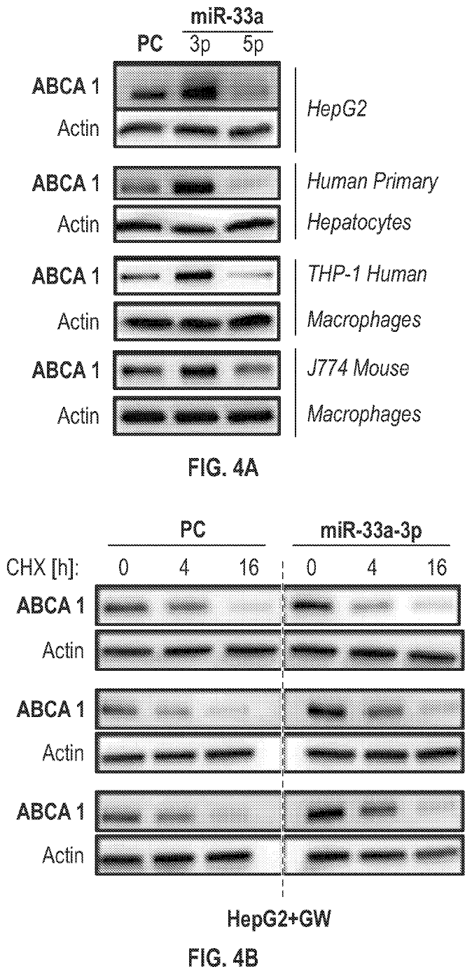

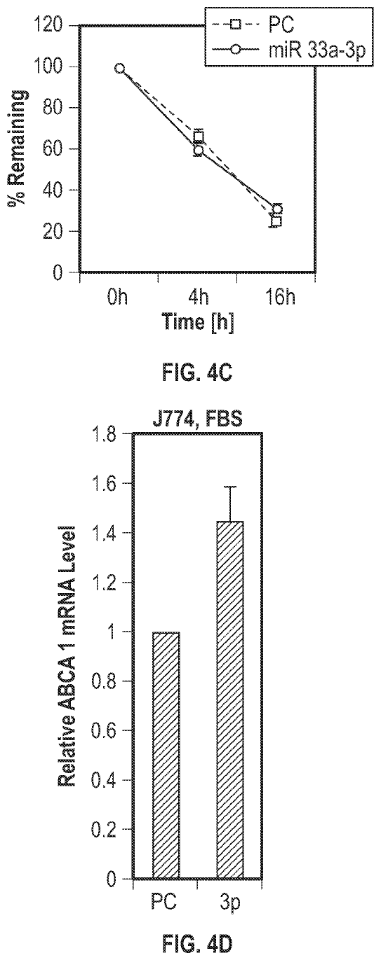

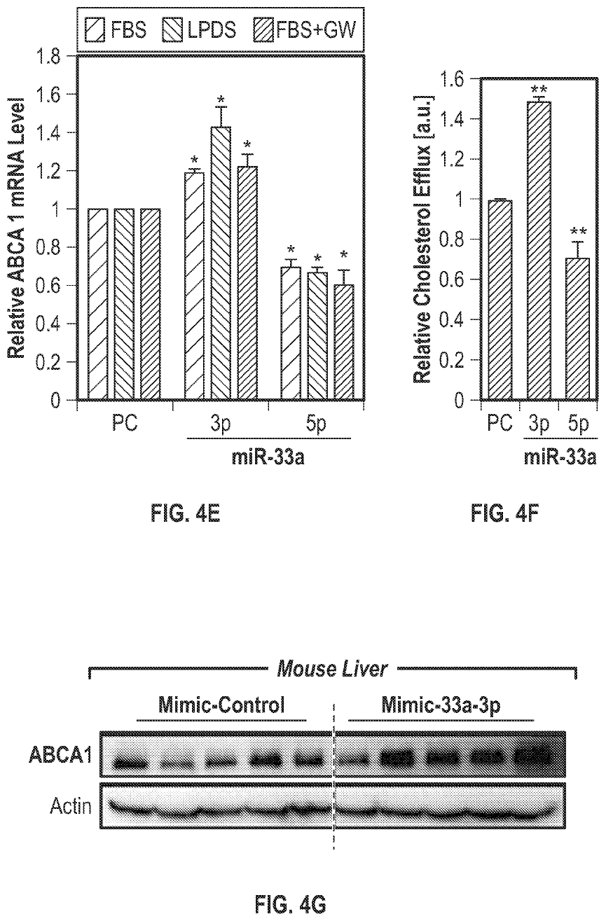

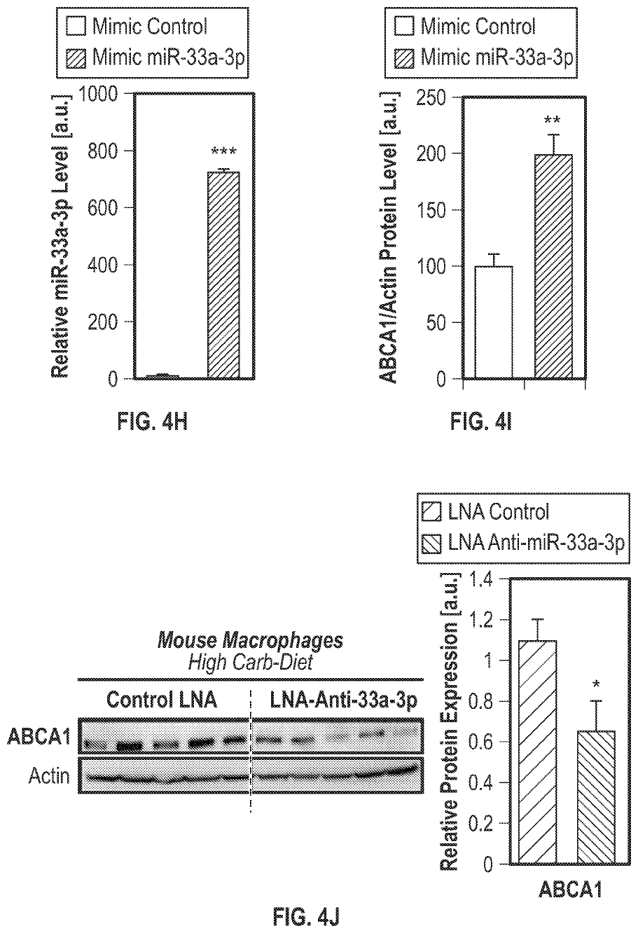

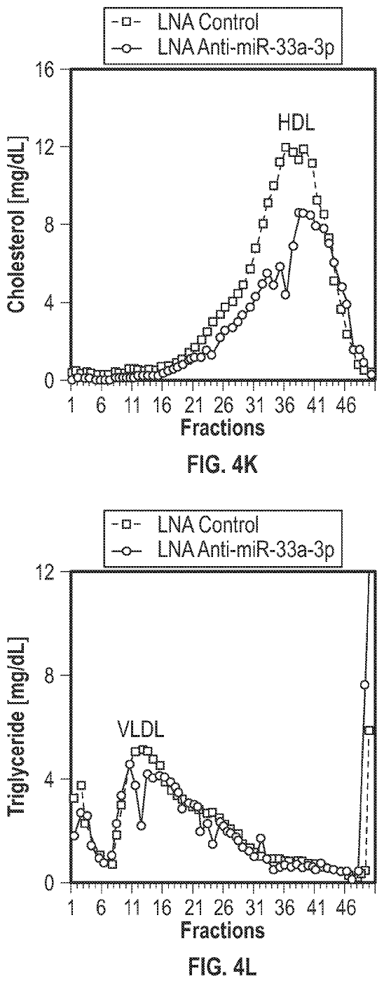

As illustrated in FIG. 4A-4B, overexpression of miR-33a-3p leads to significant increases in ABCA1 protein levels in multiple mice and human cell lines of hepatocytes and macrophages. This was surprising because miR-33a-5p has been reported to inhibit ABCA1 (Najafi-Shoushtari et al., 2010; Marquart et al., 2010).

However, ABCA1 protein levels ABCA1 protein stability was not reduced by miR-33a-3p. Instead, ABCA1 protein levels declined as miR-33a-3p levels declined after induction of miR-33a-3p by addition of cycloheximide (CHX; FIG. 4B-4C). To determine the mechanism that increases ABCA1 when miR-33a-3p is expressed, transcriptional regulation of ABCA1 was examined. Both HepG2 and J774 cells displayed an increase in ABCA1 mRNA expression upon miR-33a-3p overexpression (FIG. 4D-4E).

LDL-uptake is enhanced following miR-33a-3p overexpression that consequently may lead to increased pools of sterols which activate LXR-dependent transcription of ABCA1. Consistent with this interpretation, luciferase reporter under the transcriptional control of human ABCA1 promoter exhibited GW3965-induced expression that was further enhanced by miR-33a-3p, compared to control microRNA (data not shown). Experiments also indicated that miR-33a-3p and 5p can target genes that counteract LXR's anti-atherogenic function, including the pro-inflammatory toll-like receptor 4 (TLR4) involved in the pathogenesis of atherosclerosis, and the tetratricopeptide repeat domain protein 39B (TTC39B), that promotes LXR degradation. Notably, both genes are associated with LXR-dependent ABCA1-mediated free cholesterol efflux (Castrillo et al., 2003; Hsieh et al., 2016). It was confirmed that miR-33a-3p and 5p regulate both TLR4 and TTC39B expression in HepG2 cells with a concomitant downstream increase in LXR protein levels (data not shown).

As shown in FIG. 4F, miR-33a-3p significantly enhanced cholesterol efflux from J774 mouse macrophages. These data are distinct from the previously reported consequences of miR-33a-5p-dependent-inhibition. Furthermore, liver-specific transduction of miR-33-3p mimics into mice fed a cholesterol-rich high fat diet led to increased hepatic ABCA1 expression, as compared to the effects of a non-targeting mimic control (FIG. 4G-4I).

Conversely, LNA (Locked Nucleic Acid) antisense-mediated acute loss-of-function of miR-33-3p in mice maintained on a high-carbohydrate diet caused a reduction in ABCA1 levels in peritoneal macrophages (FIG. 4J), with a corresponding reduction in serum high-density lipoprotein (HDL)-cholesterol, but not in triglycerides (FIG. 4K-4L).

Collectively, these data show miR-33a-3p and miR-33a-5p have different roles in ABCA1 regulation and demonstrate that miR-33a-3p can reverse cholesterol transport in concert with LXR-protective functions in various cells, including macrophages.

### Example 6: miR-33a-3p Mimics Increase LDLR but Decrease PCSK9 and Total Cholesterol

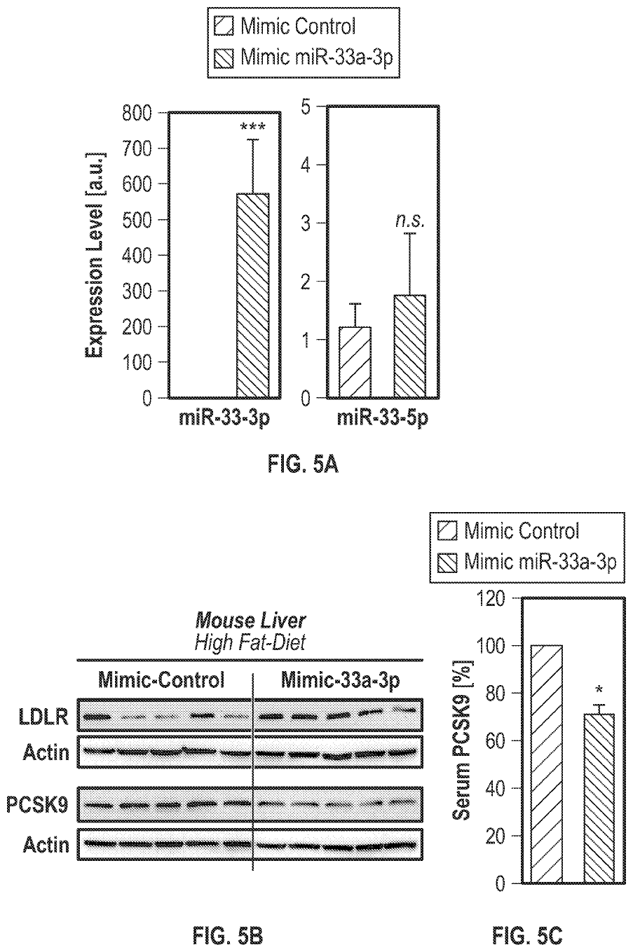

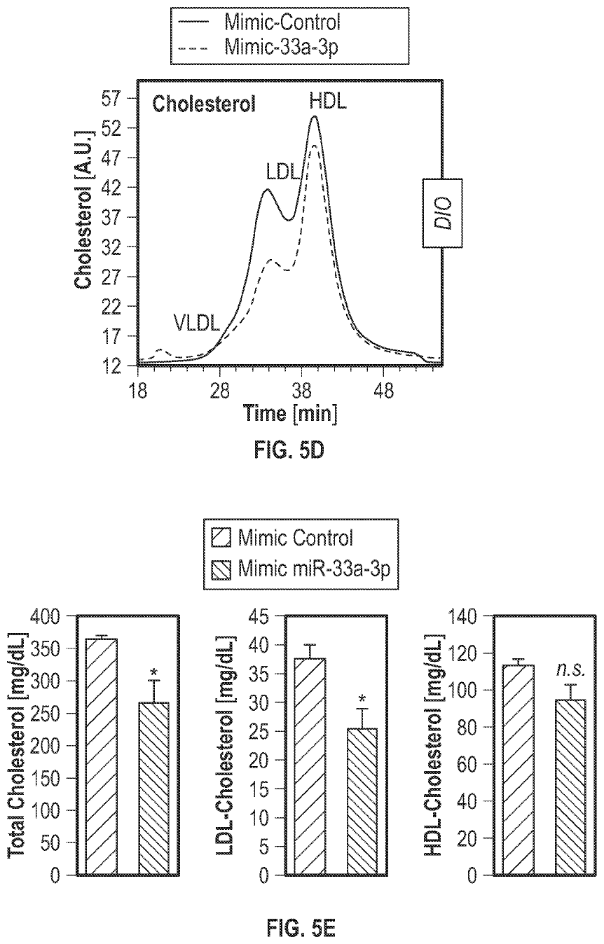

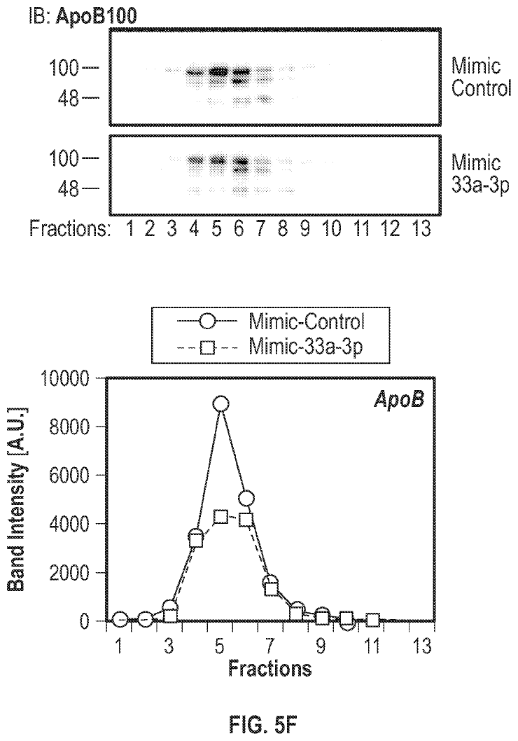

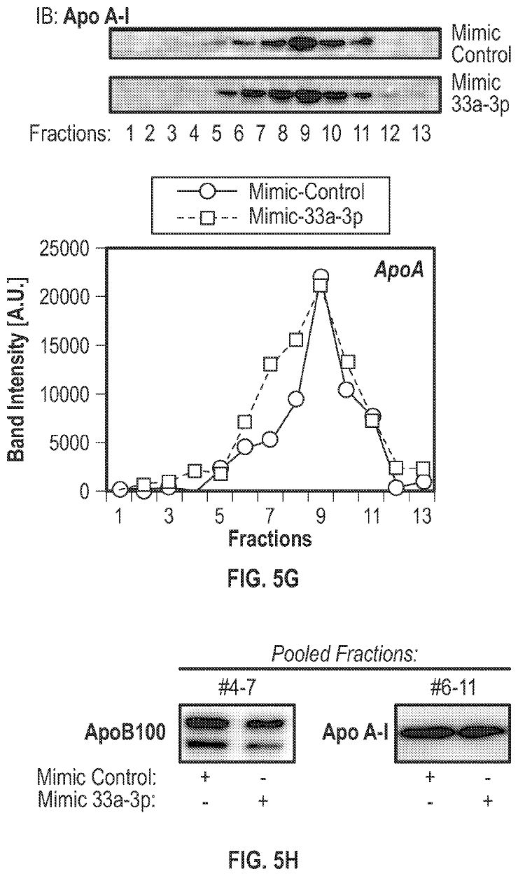

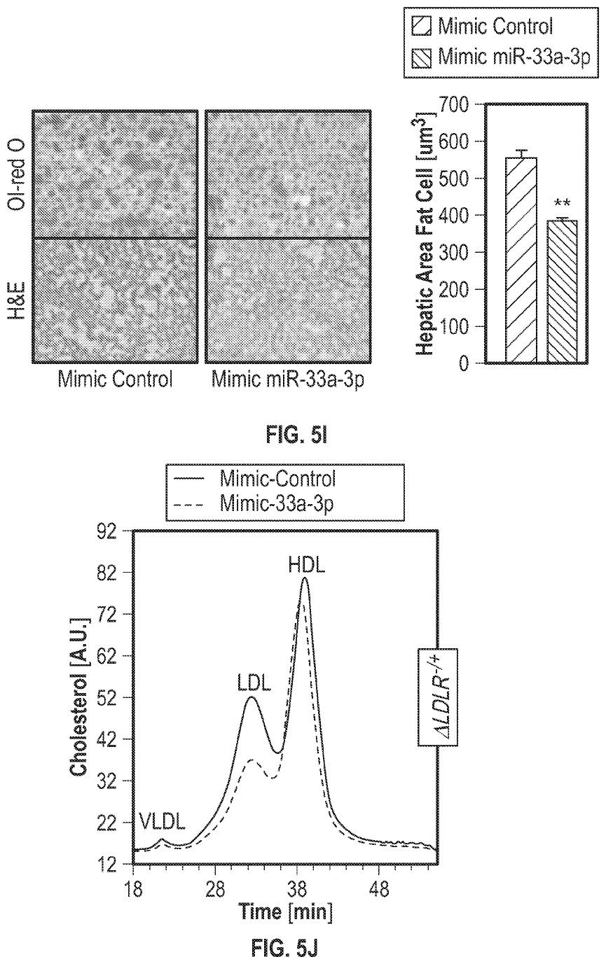

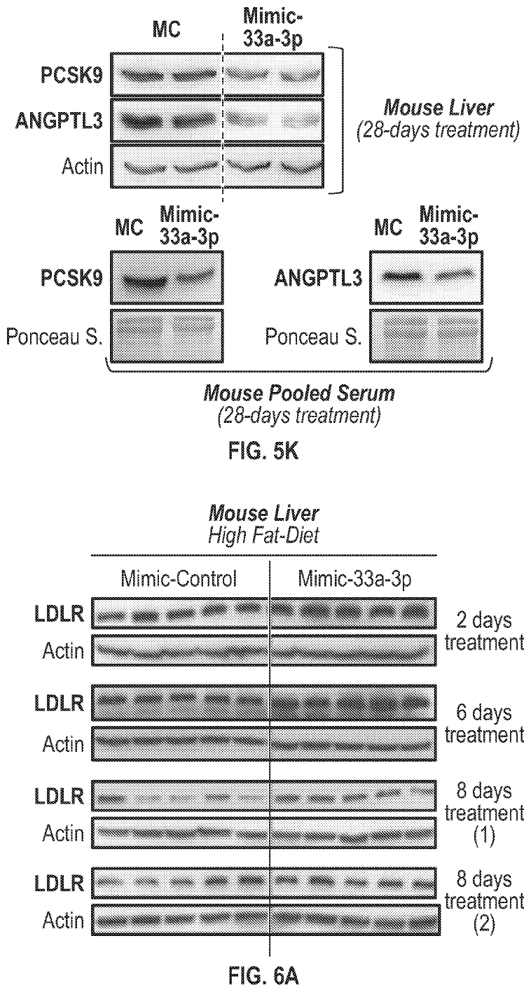

To explore miR-33a-3p function on LDLR activity in vivo, age-matched and weight-matched male C57BL/6 mice that were placed on a prolonged high-fat and cholesterol-rich diet to produce obese mice with increased serum LDL-cholesterol and hepatic steatosis. Because miR-33a-3p is largely complementary to miR-33a-5p strand, the inventors verified that the miR-33a-5p levels were unaffected by miR-33a-3p mimics (FIG. 5A). Two doses of 1 mg/kg miR-33-3p mimic were administered to the mice over eight days. As shown in FIG. 5B, decreased hepatic PCSK9 levels following strong upregulation of miR-33a-3p levels in the liver, as compared with control mimic-treated mice. Administration of the miR-33-3p mimic also led to decreased PCSK9 serum levels (FIG. 5C).

Consistent with the in vitro cell observations, in vivo administration of the miR-33-3p mimic resulted in significant increase in hepatic LDLR expression (FIG. 5B), an effect that remained consistent in all short-term studies ranging from 2 days to 8 days post-administration of the mimic. Furthermore, miR-33-3p administration decreased total cholesterol (FIG. 5D-5E), but not triglycerides. Notably, fractionation of lipoproteins revealed strongly reduced cholesterol in LDL, but not HDL, consistent with biochemical lipid profiling data (FIG. 5D-5H).

Immunoblotting of individual and pooled serum lipoprotein fractions showed that administration of the miR-33-3p mimic decreased the expression of ApoB100, but not the overall Apo A-I levels (FIG. 5F-5H), further confirming the significantly altered cholesterol distribution. No significant differences in liver function tests and serum glucose levels were observed between control and miR-33-3p mimics-treated obese mice.

As illustrated in FIG. 5I, increased VLDL secretion was correlated with a marked decrease in hepatic lipid accumulation, as shown by histological examination of livers from miR-33-3p mimic-treated mice.

In a longer term study, miR-33a-3p mimic treatment reduced PCSK9 and ANGPTL3 with concomitant decreases in plasma LDL-Cholesterol in a heterozygous knockout LDLR mice. The mice used for this study were a mouse model of familial hypercholesterolemia. As shown in FIG. 5J, cholesterol levels in LDL and HDL were reduced in the miR-33a-3p mimic-treated mice compared to control-treated mice. FIG. 5K shows that PCSK9 and ANGPTL3 levels were reduced in the livers and sera of miR-33a-3p mimic-treated mice compared to control-treated mice.

### Example 7: miR-33-3p Mimic Lowers Triglycerides and LDLs

This Examples describes experiments designed to evaluate LDLR, cholesterol, triglyceride levels in mice fed a high fat diet or a high carbohydrate diet.

In a first experiment, age-matched and weight-matched C57BL/6J DIO mice fed a high-fat diet (HFD) were injected with liver-targeting control or miR-33-3p mimics (n=5 per group). The miR-33-3p mimic was expressed for two or more days in DIO mice and hepatic LDLR, LDL-cholesterol, and LDL-associated triglycerides were measured.

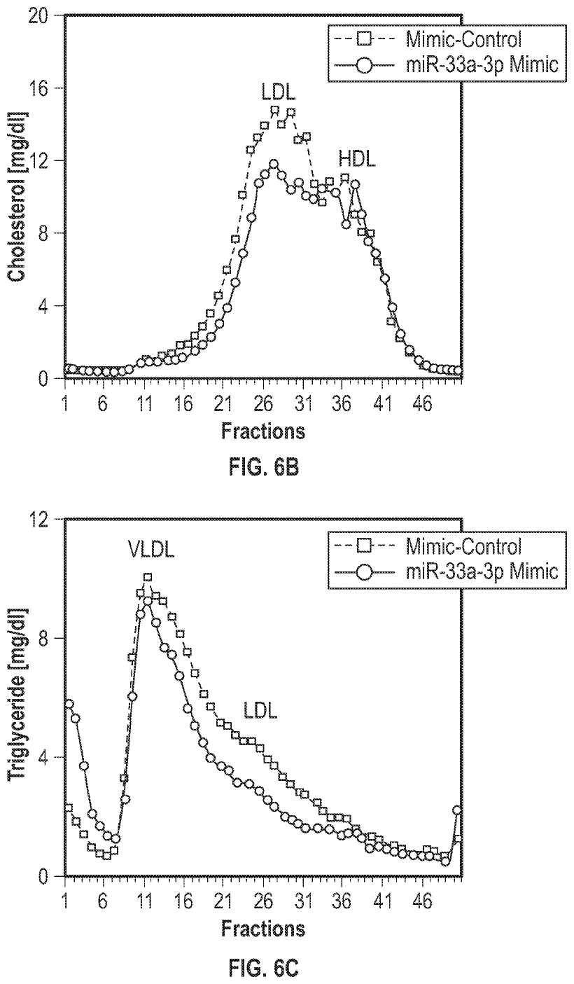

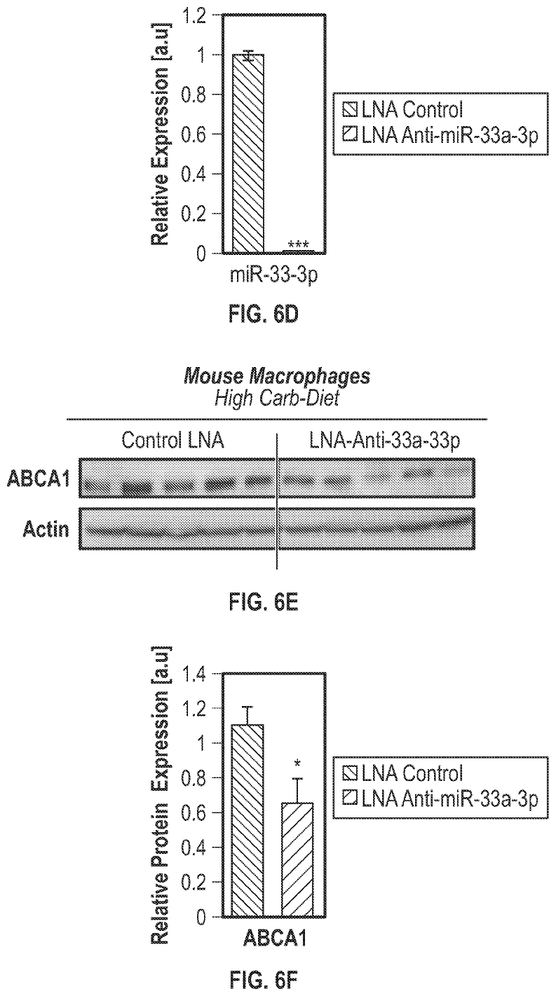

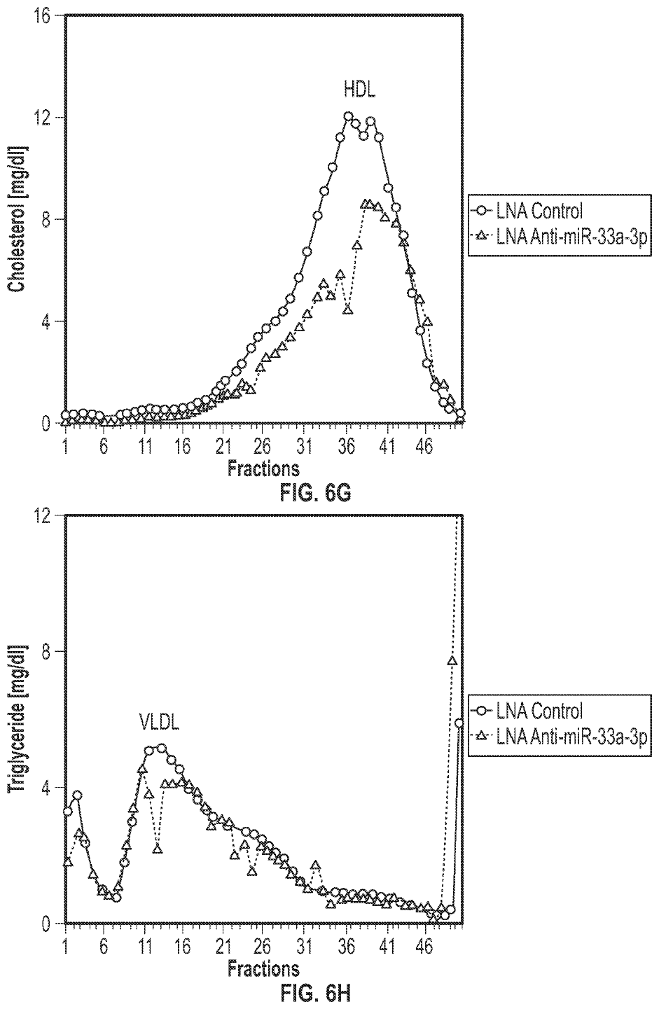

After short-term (two-day) exposure to the miR-33-3p mimic the high fat diet DIO mice consistently upregulated hepatic LDLR and lowered LDL-cholesterol and LDL-associated triglycerides. FIG. 6A illustrates that short term exposure to the miR-33-3p mimic increased hepatic LDLR levels, as detected at various time points by western blot. After two days exposure to the miR-33-3p mimic, levels of two lipoprotein were isolated by fast protein liquid chromatography (FPLC) fractionation from pooled sera of the high-fat diet mice. The peak with the earlier fractions corresponded to LDLs while the peak with the later fractions corresponded to HDLs. As shown in FIG. 6B, mice treated with the miR-33-3p mimic exhibited lower LDL-cholesterol levels (lower trace) than control mice (upper trace) who did not receive the miR-33-3p mimic. Similarly, as shown in FIG. 6C, mice exposed to the miR-33-3p mimic for two days exhibited lower levels of very low density lipoproteins (VLDL) triglycerides (lower lighter trace) compared to control mice (darker upper trace) who did not receive the miR-33-3p mimic.

In a second experiment, 24-week-old C57BL/6J DIO mice were fed a high-carbohydrate diet (HCD) and then were treated twice with antisense LNA control or antisense LNA miR-33-3p over four days (n=5 per group). FIG. 6D illustrates hepatic expression of miR-33a-3p relative to miR-423-3p as determined by quantitative RT-PCR. A western blot is shown in FIG. 6E, illustrating ABCA1 expression in peritoneal macrophages isolated from the mice treated with the antisense LNA control (left) or the antisense LNA miR-33-3p (right). FIG. 6F graphically illustrates ABCA1 expression in peritoneal macrophages isolated from the mice treated with the antisense LNA control (left bar) or the antisense LNA miR-33-3p (right bar).

FIG. 6G shows the levels of HDL-cholesterol in the serum of mice treated with the antisense LNA control (top trace) or the antisense LNA miR-33-3p (lower trace) after fractionation by fast protein liquid chromatography (FPLC) to separate the HDL from other lipoproteins. FIG. 6H shows the levels of VLDL-associated triglycerides in the serum of mice treated with the antisense LNA control (top trace) or the antisense LNA miR-33-3p (lower trace) after fractionation by fast protein liquid chromatography (FPLC) to separate the VLDL from other lipoproteins.

### Example 8: MiR-33a-3p Mimic Reduces Fat Mass; Elevates Fatty Acid Uptake by SWAT and VLDL Clearance

This Example illustrates that CETP mice exhibit reduced fat mass and elevated fatty acid uptake with VLDL clearance when treated with the miR-33-3p mimic.

CETP mice were maintained on a high fat diet and then treated once per week with the miR-33-3p mimic for five weeks. CETP mice express a Cholesteryl Ester Transfer Protein (CETP) transgene, a human CETP mini gene, and exhibit increased CEPT in the liver and plasma when maintained on a high fat diet, as well as reduced levels of plasma high density lipoproteins. Plasma VLDL and fatty acid levels were measured in various tissues, including liver, muscle, heart, spleen and adipose tissues on day 0, and after two weeks of treatment with the miR-33-3p mimic in fasted and postprandial mice.

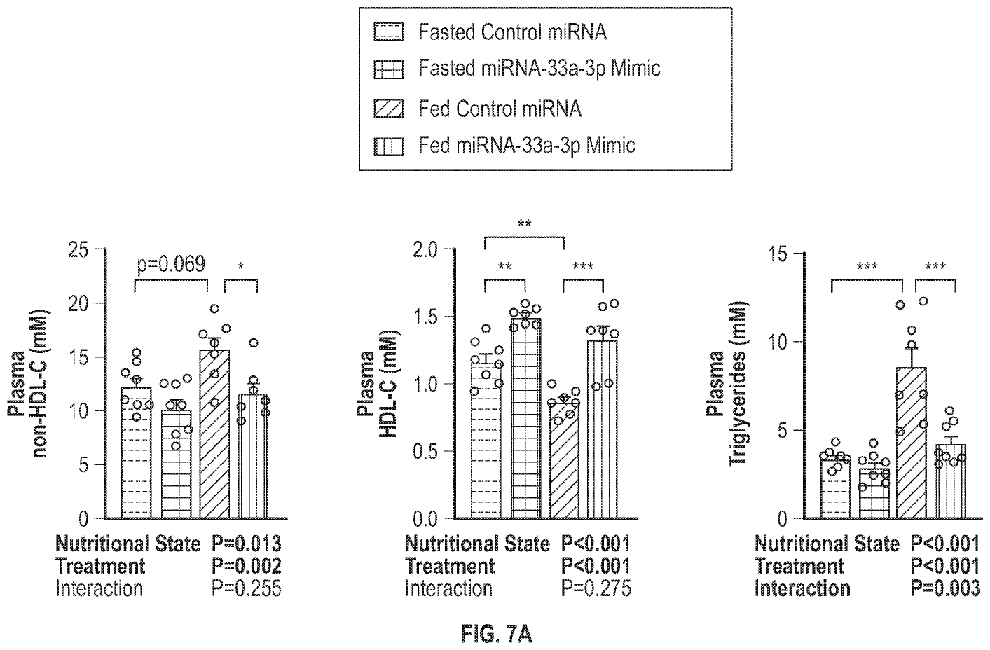

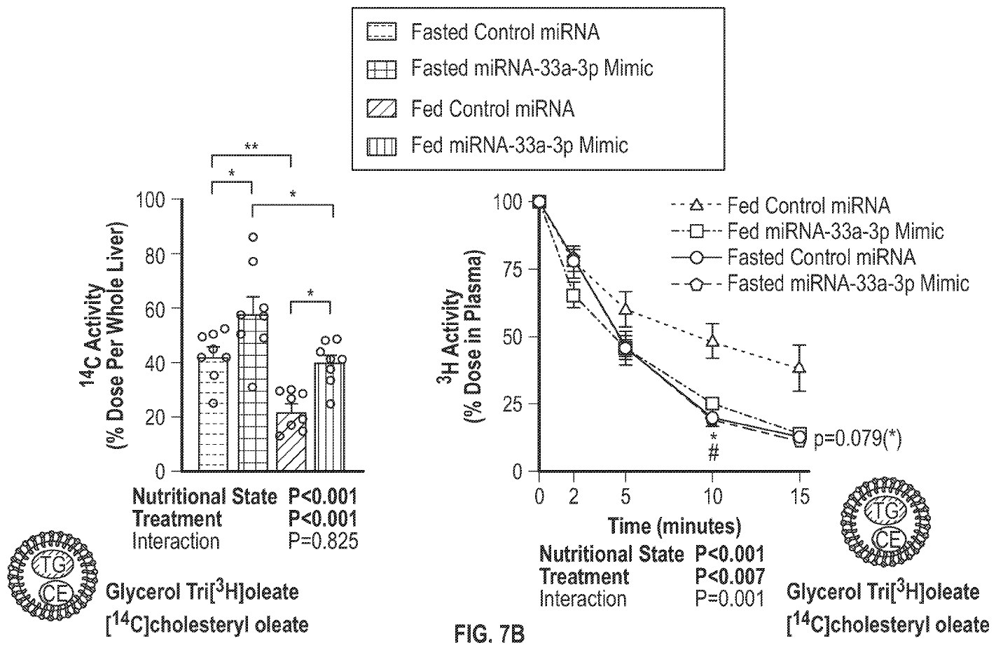

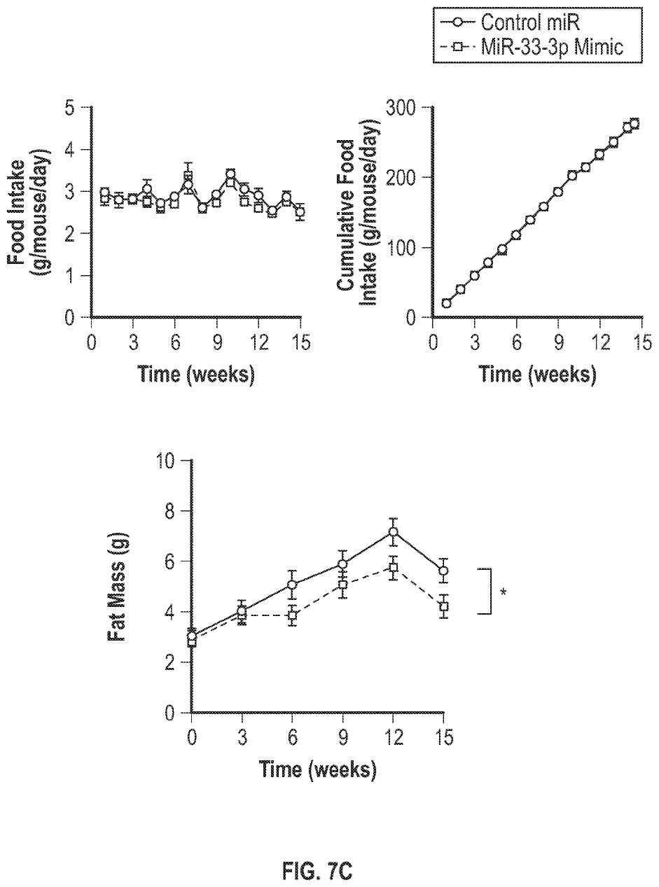

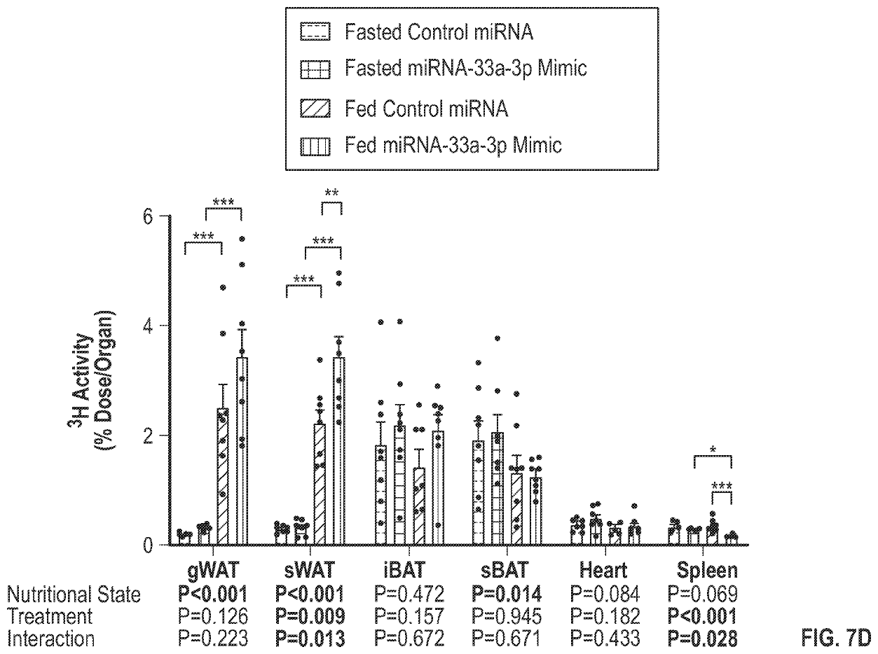

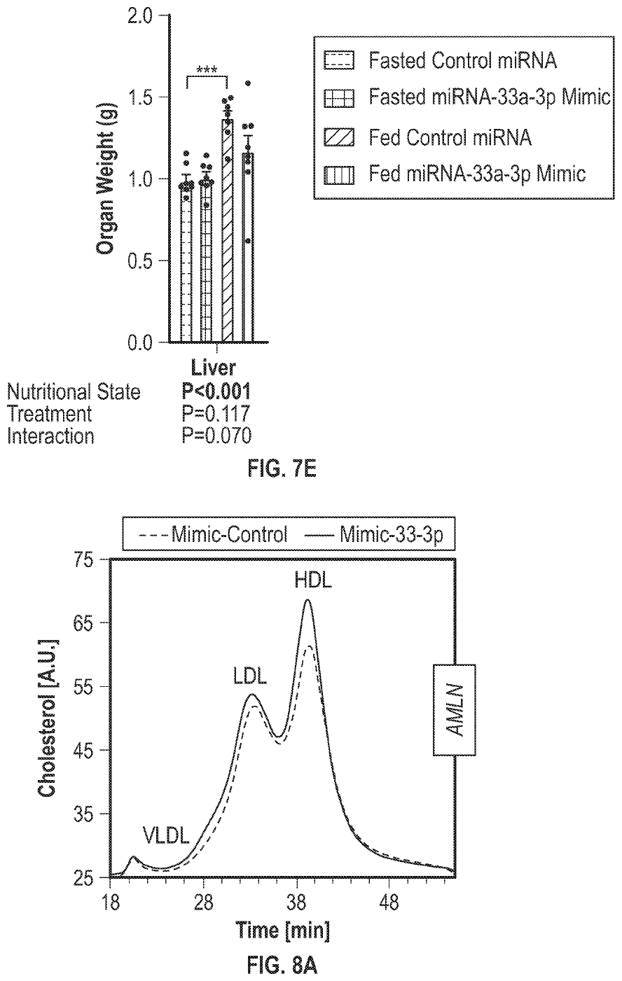

MiR-33a-3p mimic treatment elevated HDL, reduced plasma triglycerides and reduced non-HDL-C (FIG. 7A) in CETP transgenic mice. MiR-33a-3p mimic treatment also promoted VLDL clearance (FIG. 7B) and reduced fat mass in these mice without any alterations in food intake (FIG. 7C). In addition, MiR-33a-3p mimic treatment increased fatty acid uptake by sWAT (FIG. 7D) and prevented induced liver weight gain that can occur as a consequence of ER-stress and increased lipogenesis at postprandial state (FIG. 7E).

During fasting, white adipose tissue (WAT) normally releases fatty acids which are taken up by oxidative tissues as a source of energy. Upon refeeding, however, the flux of fatty acids is reversed and the reservoirs of triglyceride in WAT are replenished, primarily from circulating lipoproteins such as VLDL from liver and chylomicrons from the intestines. The data indicate that miR-33a-3p mimics increase VLDL-triglyceride uptake by WATs upon refeeding, reflecting increased LPL activity (FIG. 7B). MiR-33a-3p mimic treatment therefore reduced fat mass (FIG. 7C). And these beneficial effects were achieved without alteration in food intake by the miR-33a-3p mimic treated CETP mice (FIG. 7C).

### Example 9: MiR-33a-3p Mimic Improves LDL/HDL Ratio in Mice with Non-Alcoholic Liver Disease

Nonalcoholic steatohepatitis (NASH) has become a major cause of cirrhosis and liver-related deaths worldwide. NASH is strongly associated with obesity and the metabolic syndrome, conditions that cause lipid accumulation in hepatocytes (hepatic steatosis). It is not well understood why some, but not other, individuals with hepatic steatosis develop NASH. The factors that determine whether or not NASH progresses to cirrhosis are also unclear (Suzuki et al., 2017).

This Example illustrates that miR-33a-3p mimic treatment can improve non-alcoholic liver disease in mice fed a high-fat/high-fructose/high-cholesterol diet.

Mice were fed a high-fat, high-fructose, and high-cholesterol Amylin liver NASH (AMLN) diet to induce non-alcoholic steatohepatitis (NASH). The mice were then administered either the miR-33a-3p mimic or a non-miR-33a-3p mimic control.

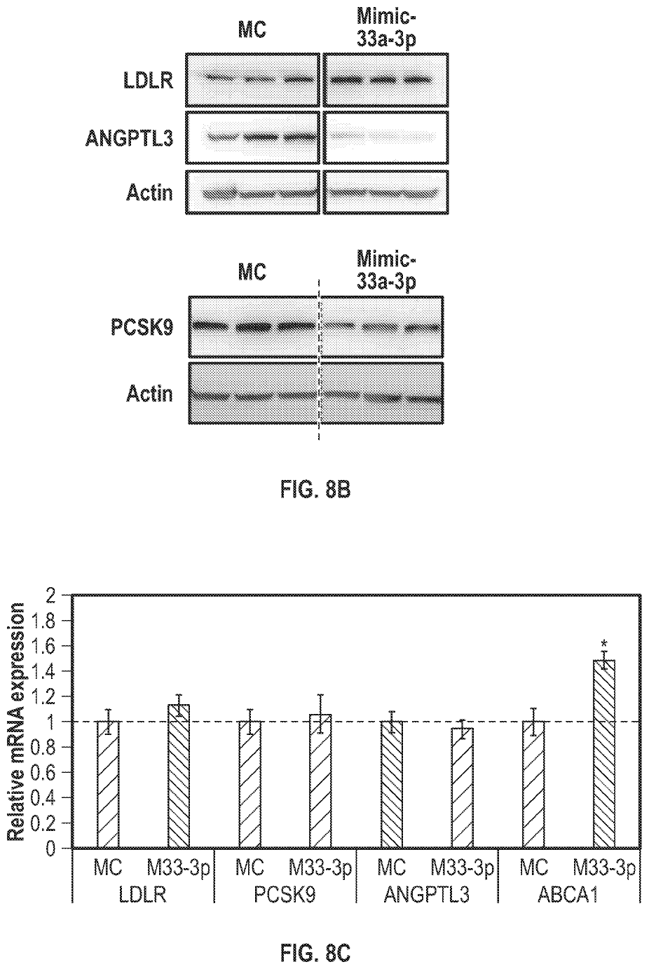

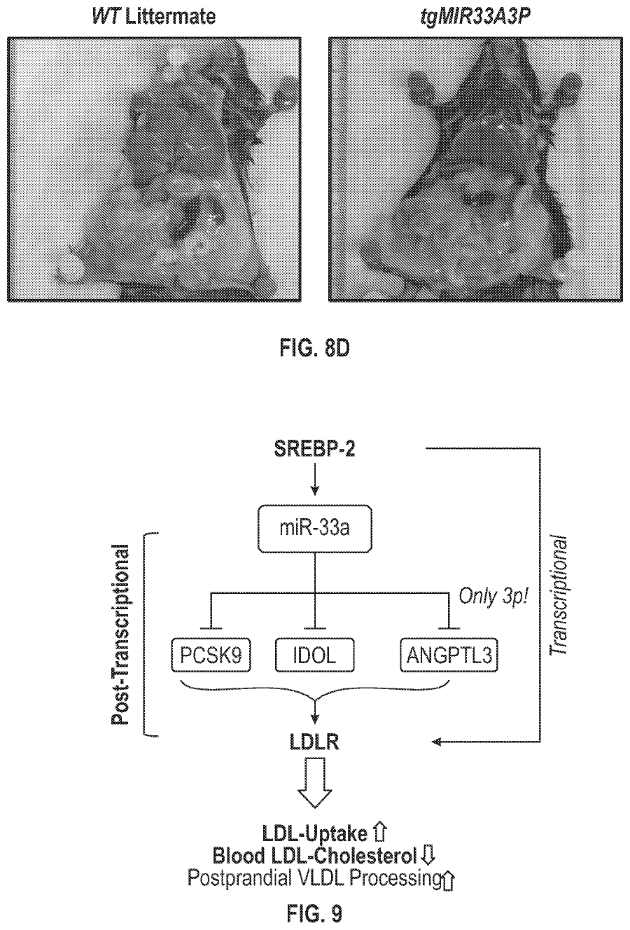

FIG. 8 illustrates that miR-33a-3p mimic treatment improves LDL/HDL ratios in the NASH mice fed a high-fat/high-fructose/high cholesterol diet. FIG. 8A illustrates cholesterol levels of VLDL, LDL, and HDL lipoproteins of NASH mice treated with the miR-33a-3p mimic or the non-treated control (mimic control). FIG. 8B shows immunoblots illustrating LDLR, ANGPTL3, and PCSK9 protein levels in NASH mice treated with the miR-33a-3p mimic or the non-treated control (MC, mimic control). FIG. 8C graphically illustrates relative RNA levels expressed by LDLR, PCSK9, ANGPTL3, ABCA1 genes of the miR-33a-3p mimic or the non-treated control (MC, mimic control) mice. FIG. 8D shows representative images of a wild type (untreated) mouse and a transgenic miR-33a-3p mouse, after both were kept on a 60% high fat diet for 12 weeks, showing that the liver of the transgenic miR-33a-3p mouse is healthy and red, indicating that it was protected from developing hepatic steatosis, a major event associated with NASH. n=3-5.

### Example 9: MiR-33a-3p Mimic Beneficially Alters NASH/NAFLD Gene Expression

MiR-33-3p mimics promotes fatty acid beta oxidation in obese mouse livers. Global analysis of hepatic gene expression profiles between the two groups of mice revealed an enrichment towards activated genes involved in fatty acid beta-oxidation pathway (Acsbg1, Cptla, Acadvl, Acsm3, Echdc1, and Acad11) and a battery of other metabolic genes that contribute to liver triglyceride content and protect against nonalcoholic steatohepatitis (i.e. SORT1, KFL4, LPIN1).

An RNA-seq analysis was performed on liver samples after 3p mimic treatment of Leiden CETP transgenic mice kept on a western diet. Table 2 lists hepatically expressed genes associated with NASH/NAFLD that exhibited beneficial expression profiles after such miR-33a-3p mimic treatment.

TABLE 2
Genes Modulated by MiR-33-3p mimics
associated with NAFLD/NASH

| | Log2Fold | | Function related to |
|---|---|---|---|
| gene name | Change | p-value | NASH/NAFLD |
| Cxcl1 | −1.228720858 | 6.41E−09 | inflammation marker |
| | | | (key chemokine for |
| | | | neutrophil infiltration, |
| | | | a hallmark of NASH) |
| Pnpla3 | −1.228471808 | 0.002535802 | NASH Therapeutic target |
| Srebf1 | −0.950071664 | 4.67E−07 | Increased in NASH |
| Fitm1 | −0.926920707 | 0.000336157 | promotes fat storage |
| Slc10a1 | −0.845388965 | 2.86E−18 | bile acid transporter |
| | | | protects against |
| | | | hepatic steatosis when |
| | | | reduced |
| Cxcl9 | −0.842874353 | 0.000311358 | inflammation marker |
| Cd86 | −0.773188204 | 5.12E−06 | inflammation marker |
| Trem2 | −0.698228888 | 0.003592643 | Increased in NASH |
| Tlr4 | −0.437950643 | 0.002027839 | Increased in NASH |
| Tram1 | −0.329593573 | 4.71E−05 | Binds to TLR4 |
| Tspo | −0.324047893 | 5.27E−05 | inhibition reduces NASH |
| Xbp1 | −0.221013639 | 0.006567244 | ER-stress Marker, elevated |
| | | | in hepatic steatosis |
| Eng | 0.240189281 | 0.001026716 | reduced in NASH |
| Lrp1 | 0.252080929 | 0.008613923 | reduced in NASH |
| Pdgfrb | 0.516874056 | 0.000177551 | reduced in NASH |
| Tcf21 | 0.55487423 | 0.009086315 | reduces NASH |

### REFERENCES

Abifadel et al., Nature Gen., 34:154 (2003).
Brummer & Hausser, BioEssays, 36:617 (2014).
Castrillo et al., Molecular Cell, 12:805 (2003).
Corcoran et al., Genome Bio., 12: R79 (2011).
Fairall et al., Proc. Natl. Acad. Sci., (2011).
Gaudet et al., N. Engl. J. Med., 377:222 (2017).
Goldstein & Brown, Arterio. Thrombosis Vasc. Biol., 29: 431 (2009).
Hafner et al., Cell, 141:129 (2010).
Hafner et al., J. Visual. Exp., 41, pii 2034 (2010).
Hong et al., Cell Metab., 20:910 (2014).
Horton et al., J. Clin. Invest., 101:2331 (1998).
Horton et al., PNAS, 100:12027 (2003).
Hsieh et al., Nature, 535:303 (2016).
Hsieh et al., Nature, 535:303 (2016).
Hua et al., PNAS, 90:11603 (1993).
Kathiresan et al., Nat. Genet., 40:189 (2008).
Kumar et al., Bioorg. Med. Chem. Lett., 8:2219 (1998).
Lu et al., Cell Rep., 9:2330 (2014).
Madison, J. Lipid Res., 57:333 (2016).
Marquart et al., Proc. Natl. Acad. Sci., 107:12228 (2010).
Mehlem et al., Nature Protocols, 8:1149 (2013).
Musunuru et al., N. Engl. J. Med., 363:2220 (2010).
Musunuru et al., Nat. Rev. Endocrinol., 13:503 (2017).
Najafi-Shoushtari et al., Science, 328:1566 (2010).
Navab et al., Nature Rev. Cardio., 8:222 (2011).
Page Watts, Diab. Obes. Metab., 20:270 (2018).
Phillips, J. Lipid Res., 59:749 (2018).
Scotti et al., Mol. Cell. Biol., 31:1885 (2011).
Scotti et al., Mol. Cell. Biol., 33:1503 (2013).
Seidah et al., Circul. Res., 114:1022 (2014).
Sharifnia et al., Am. J. Physiol. Gastrointest. Liver Physiol., 309: G270 (2015).
Singh et al., J. Org. Chem., 63:10035 (1998).
Sorrentino et al., J. Lipid Res., 54:2174 (2013).
Suzuki et al., Annu. Rev. Med., 68:85 (2017).
Wang & Tontonoz, Nature Rev. Endocr., 14:452 (2018).
Weischenfeldt & Porse, CSH Protocols, December 1, prot5080 (2008).
Xu et al., Atherosclerosis, 268:196 (2018).
Zelcer et al., Science, 325:100 (2009).
Zhang et al., Arterio. Thrombosis Vasc. Biol., 32:2541 (2012).
Zhao et al., Adv. Genet., 88:13 (2014).

All publications, patents and patent applications are incorporated herein by reference. While in the foregoing specification, this invention has been described in relation to certain embodiments thereof, and many details have been set forth for purposes of illustration, it will be apparent to those skilled in the art that the invention is susceptible to additional embodiments and that certain of the details herein may be varied considerably without departing from the basic principles of the invention.

### STATEMENTS

1. A method to prevent, inhibit or treat liver disease in a mammal, comprising administering to a mammal in need thereof an effective amount of a composition comprising a nucleic acid sequence comprising a seed region of miRNA-33a-3p.

2. The method of statement 1, wherein the mammal is a human.

3. The method of statement 1 or 2, wherein the disease is steatosis, non-alcoholic fatty liver disease (NAFLD), nonalcoholic steatohepatitis (NASH), or cardiovascular disease.

4. The method of statement 1 or 2, wherein the mammal has alcohol fatty liver disease or chronic liver disease.

5. The method of any one of statements 1 to 4, wherein the composition comprises liposomes.

6. The method of statement 5, wherein the liposomes comprise or more of DC-cholesterol, 1,2-dioleoyl-sn-glycero-3-phosphoethanolamine (DOPE), an ionizable cationic lipid or a lipidoid.

7. The method of any one of statements 1 to 6, wherein the composition comprises nanoparticles.

8. The method of any one of statements 1 to 7, wherein the composition is targeted to the liver.

9. The method of statement 8, wherein the composition comprises collagen type VI receptor, mannose-6-phosphate, galactose or asialoglycoprotein.

10. The method of any one of statements 1 to 9, wherein the composition is systemically administered.

11. The method of any one of statements 1 to 9, wherein the composition is orally administered.

12. The method of any one of statements 1 to 10, wherein the composition is injected.

13. The method of any one of statements 1 to 12, wherein the seed region comprises 5′AAUGUUU3′ or 5′AATGTTT3′; 5′CAAUGUUU3′ or 5′CAATGTTT.′

14. The method of any one of statements 1 to 13, wherein the nucleic acid sequence is less than 30 bases in length.

15. The method of any one of statements 1 to 13 wherein the nucleic acid sequence is less than 25 bases in length.

16. The method of any one of statements 1 to 13, wherein the nucleic acid sequence is less than 20 bases in length.

17. The method of any one of statements 1 to 13, wherein the nucleic acid sequence is greater than 10 bases in length.

18. The method of any one of statements 1 to 17, wherein the composition comprises single stranded RNA comprising the seed region.

19. The method of any one of statements 1 to 18, wherein the composition comprises RNA comprising a hairpin-loop structure.

20. The method of any one of statements 1 to 19, wherein the composition comprises double stranded nucleic acid comprising the seed region.

21. The method of statement 18, 19 or 20, wherein the RNA or one strand of the double stranded nucleic acid is less than 70 bases in length.

22. The method of any one of statement 18-21, wherein the RNA or one strand of the double stranded nucleic acid is less than 50 bases in length.

23. The method of any one of statement 18-22, wherein the RNA or one strand of the double stranded nucleic acid is less than 40 bases in length.

24. The method of any one of statement 18-23, wherein the RNA or one strand of the double stranded nucleic acid is less than 25 bases in length.

25. The method of any one of statement 18-24, wherein the RNA or one strand of the double stranded nucleic acid is greater than 10 bases in length.

26. The method of any one of statement 18-25, wherein the length of the one strand is greater than that of the nucleic acid sequence having the seed region.

27. The method of any one of statement 18-26, wherein the RNA or the one strand is linked to a molecule that enhances cellular uptake.

28. The method of any one of statement 18-27, wherein the nucleic acid sequence comprises non-native nucleotides.

29. The method of statement 28, wherein the non-native nucleotides have a modified nucleobase, modified phosphate group, a modified sugar, or a combination thereof.

30. The method of any one of statements 18 to 29, wherein the RNA or the one strand comprises non-native nucleotides.

31. The method of statement 30, wherein the non-native nucleotide has a modified nucleobase, modified phosphate group or a modified sugar.

32. The method of any one of statements 1 to 31, wherein the nucleic acid sequence comprising a seed region of miRNA-33a-3p does not comprise miR-33b sequences or miRNA-33a-5p sequences.

33. The method of any one of statements 1 to 32, wherein the amount of the nucleic acid sequence is about 0.01 mg/kg to about 100 mg/kg.

34. The method of any one of statements 1 to 33, wherein the amount of the nucleic acid sequence is about 0.05 mg/kg to about 10 mg/kg.

35. The method of any one of statements 1 to 34, wherein the amount of the nucleic acid sequence is about 10 mg/kg to about 75 mg/kg.

36. The method of any one of statements 1 to 35 wherein the amount of the nucleic acid sequence is about 1 mg/kg to about 100 mg/kg.

37. A liver targeted composition comprising a nucleic acid sequence comprising a seed region of miRNA-33a-3p.

38. The composition of statement 37, wherein the composition comprises nanoparticles.

39. The composition of statement 37 or 38, wherein the composition comprises one or more distinct lipids.

40. The composition of any one of statements 37 to 39, which comprises a liver targeted molecule conjugated to the particles or one of the one or more lipids.

41. The composition of any one of statements 37 to 40, wherein the nucleic acid sequence comprises one or more modified phosphodiester bonds.

42. The composition of statements 41, wherein the modification is a phosphorothioate bond.

43. The composition of any one of statements 37 to 42, wherein the nucleic acid sequence comprises one or more modified nucleotides.

44. The composition of statements 43, wherein the modification is 2′-O-methyl, 2′-O-methoxyethyl, 2′-fluoro, locked nucleic acid (LNA), or 5′ vinylphosphonate.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also forms part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

1. A method to prevent, inhibit or treat liver disease in a mammal, comprising: administering to a mammal in need thereof an effective amount of a composition comprising a nucleic acid sequence comprising a seed region of miRNA-33a-3p.

2. The method of claim 1, wherein the mammal is a human.

3. The method of claim 1, wherein the disease is steatosis, non-alcoholic fatty liver disease NAFLD), nonalcoholic steatohepatitis (NASH), or cardiovascular disease.

4. The method of claim 1, wherein the mammal has alcohol fatty liver disease or chronic liver disease.

5. The method of claim 1, wherein the composition comprises liposomes.

6. The method of claim 5, wherein the liposomes comprise or more of DC-cholesterol, 1,2-dioleoyl-sn-glycero-3-phosphoethanolamine (DOPE), an ionizable cationic lipid or a lipidoid.

7. The method of claim 1, wherein the composition comprises nanoparticles.

8. The method of claim 1, wherein the composition is targeted to the liver.

9. The method of claim 8, wherein the composition comprises collagen type VI receptor, mannose-6-phosphate, galactose or asialoglycoprotein.

10. The method of claim 1, wherein the composition is systemically administered, orally administered, or injected.

11. (canceled)

12. (canceled)

13. The method of claim 1, wherein the seed region comprises S′AAUGUUU3′ or S′AATGTTT3′; 5′CAAUGUUU3′ or 5′CAATGTTT.′

14. The method of claim 1, wherein the nucleic acid sequence is less than 30 bases in length.

15. The method of claim 1, wherein the nucleic acid sequence is less than 25 bases in length.

16. The method of claim 1, wherein the nucleic acid sequence is less than 20 bases in length.

17. The method of claim 1, wherein the nucleic acid sequence is greater than 10 bases in length.

18. The method of claim 1, wherein the composition comprises single stranded RNA comprising the seed region.

19. The method of claim 1, wherein the composition comprises RNA comprising a hairpin-loop structure, or a double stranded nucleic acid comprising the seed region.

20. The method of claim 19, wherein the RNA or one strand of the double stranded nucleic acid is less than 70 bases in length, less than 50 bases in length less than 40 bases in length, or less than 25 bases in length.

21. (canceled)

22. (canceled)

23. (canceled)

24. The method of claim 19, wherein the RNA or one strand of the double stranded nucleic acid is greater than 10 bases in length.

25. The method of claim 19, wherein the length of the one strand is greater than that of the nucleic acid sequence having the seed region.

26. The method of claim 19, wherein the RNA or the one strand is linked to a molecule that enhances cellular uptake.

27. The method of claim 19, wherein the nucleic acid sequence comprises non-native nucleotides.

28. The method of claim 27, wherein the non-native nucleotide has a modified nucleobase, modified phosphate group or a modified sugar.

29. The method of claim 19, wherein the RNA or the one strand of the double stranded nucleic acid comprises non-native nucleotides.

30. The method of claim 29, wherein the non-native nucleotide has a modified nucleobase, modified phosphate group or a modified sugar.

31. The method of claim 1, wherein the nucleic acid sequence comprising a seed region of miRNA-33a-3p does not comprise miR-33b sequences or miRNA-33a-5p sequences.

32. The method of claim 1, wherein the amount of the nucleic acid sequence is about 0.01 mg/kg to about 100 mg/kg, about 0.05 mg/kg to about 10 mg/kg, about 10 mg/kg to about 75 mg/k, or about 1 mg/kg to about 100 mg/kg.

33. (canceled)

34. (canceled)

35. (canceled)

36. A liver targeted composition comprising a nucleic acid sequence comprising a seed region of miRNA-33a-3p.

37. The composition of claim 36, wherein the composition comprises nanoparticles.

38. The composition of claim 36, wherein the composition comprises one or more distinct lipids.

39. The composition of claim 38, which comprises a liver targeted molecule conjugated to the particles or one of the one or more lipids.

40. The composition of claim 36, wherein the nucleic acid sequence comprises one or more modified phosphodiester bonds.

41. The composition of claim 36, wherein the nucleic acid sequence comprises one or more phosphorothioate bond.

42. The composition of claim 36, wherein the nucleic acid sequence comprises one or more modified nucleotides.

43. The composition of claim 42, wherein the modification is 2′-O-methyl, 2′-O-methoxyethyl, 2′-fluoro, locked nucleic acid (LNA), or 5′ vinylphosphonate.
